(19) United States
(12) Patent Application Publication
(10) Pub. No.: US 2007/0141684 A1
(43) Pub. Date: Jun. 21, 2007

(54) Preparation of gamma-amino acids having affinity for the alpha-2-delta protein

(75) Inventors: Margaret Claire Evans, Kalamazoo, MI (US); Lloyd Charles Franklin, Howell, MI (US); Lorraine Michelle Murtagh, Sandwich (GB); Thomas Norman Nanninga, Kalamazoo, MI (US); Bruce Allen Pearlman, Westfield, NJ (US); James Edward Saenz, Ypsilanti, MI (US); Niamh Josephine Willis, Sittingbourne (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/636,304

(22) Filed: Dec. 8, 2006

Related U.S. Application Data
(60) Provisional application No. 60/752,839, filed on Dec. 21, 2005.

(51) Int. Cl. C12P 13/00; C07C 255/17

(52) U.S. Cl. 435/128; 558/410; 558/441

(57) ABSTRACT

Disclosed are materials and methods for preparing optically active γ-amino acids of Formula 1, 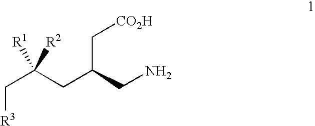 which bind to the alpha-2-delta (α2δ) subunit of a calcium channel.

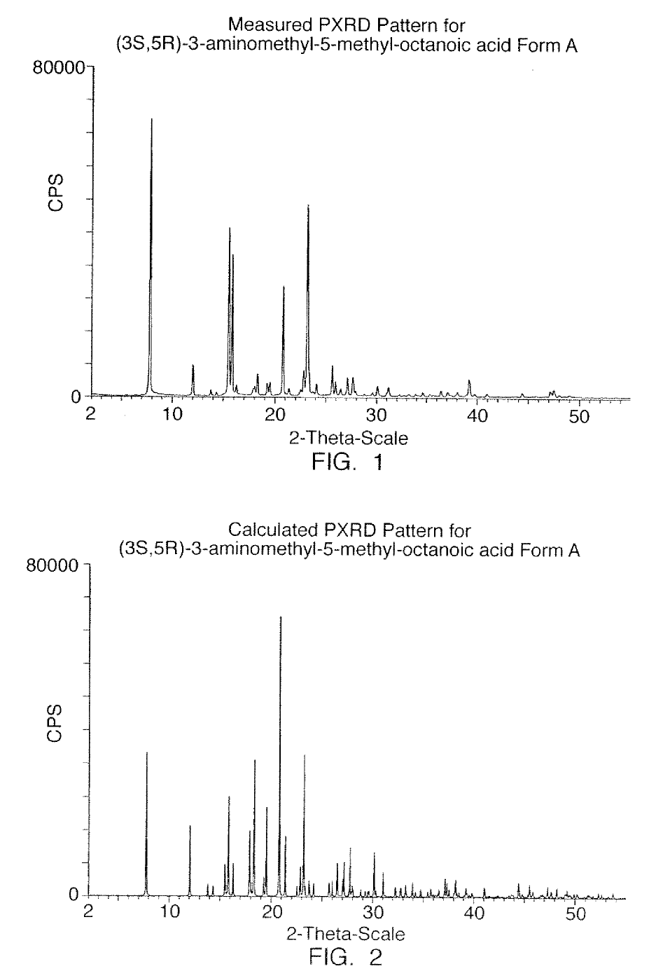

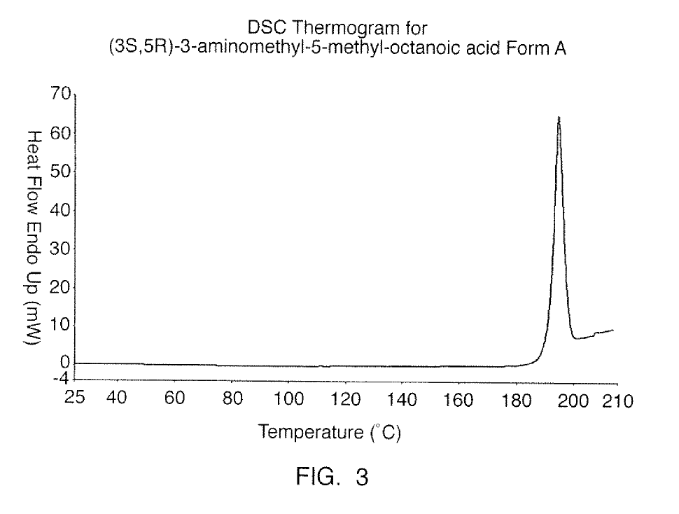

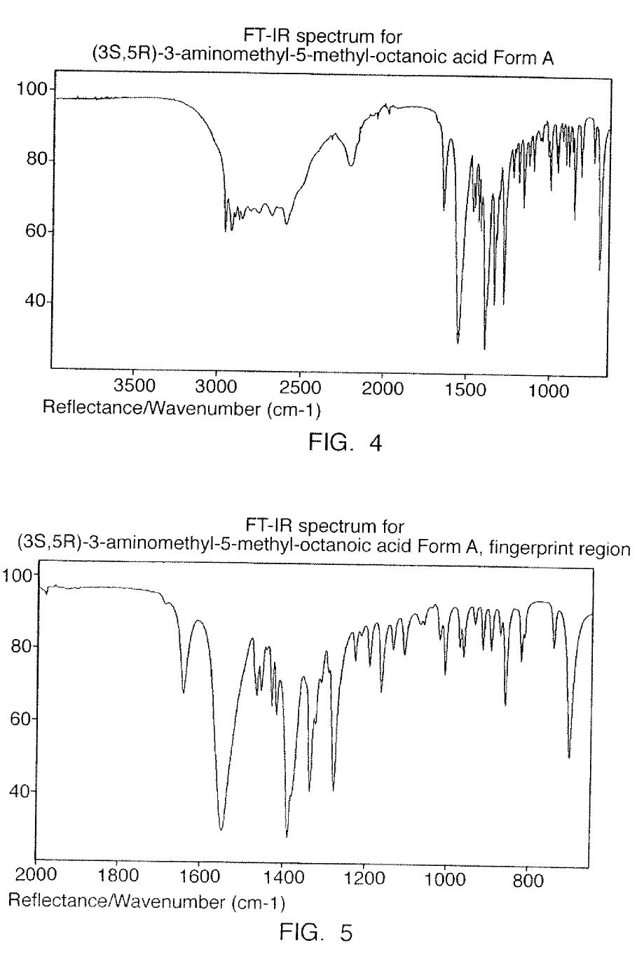

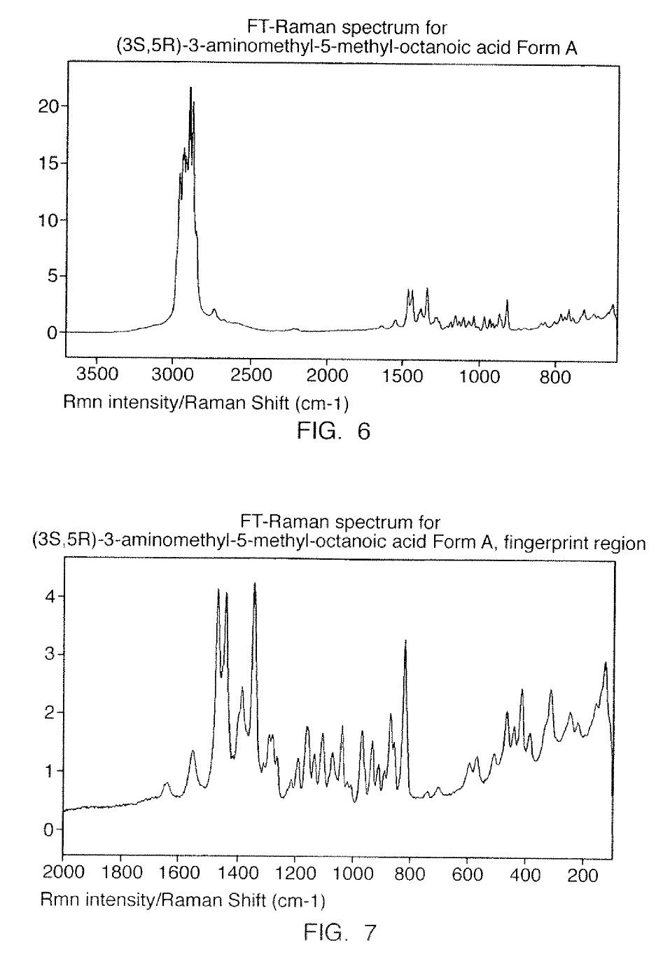

### BACKGROUND OF THE INVENTION

### FIELD OF INVENTION

This invention relates to materials and methods for preparing optically-active γ-amino acids that bind to the alpha-2-delta (α2δ) subunit of a calcium channel. These compounds, including their pharmaceutically acceptable complexes, salts, solvates and hydrates, are useful for treating epilepsy, pain, and a variety of neurodegenerative, psychiatric and sleep disorders.

### DISCUSSION

U.S. Pat. No. 6,642,398 to Belliotti et al. (the '398 patent) describes γ-amino acids that bind to the γ2δ subunit of a calcium channel. These compounds, along with their pharmaceutically acceptable complexes, salts, solvates, and hydrates, may be used to treat a number of disorders, medical conditions, and diseases, including, among others, epilepsy; pain (e.g., acute and chronic pain, neuropathic pain, and psychogenic pain); neurodegenerative disorders (e.g., acute brain injury arising from stroke, head trauma, and asphyxia); psychiatric disorders (e.g., anxiety and depression); and sleep disorders (e.g., insomnia, drug-associated sleeplessness, hypersomnia, narcolepsy, sleep apnea, and parasomnias).

Many of the γ-amino acids described in the '398 patent are optically active. Some of the compounds, like those represented by Formula 1, below, possess two or more stereogenic (chiral) centers, which make their preparation challenging. Although the '398 patent describes useful methods for preparing optically-active γ-amino acids, some of the methods may be problematic for pilot- or full-scale production because of efficiency or cost concerns. Thus, improved methods for preparing optically-active γ-amino acids, including those given by Formula 1, would be desirable.

### SUMMARY OF THE INVENTION

The present invention provides comparatively efficient and cost-effective methods for preparing compounds of Formula 1, 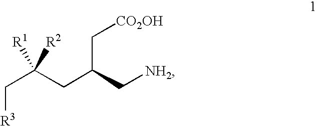
or a diastereomer thereof or a pharmaceutically acceptable complex, salt, solvate or hydrate thereof, wherein:

R¹ and R² are each independently selected from hydrogen atom and C₁₋₃ alkyl, provided that when R¹ is a hydrogen atom, R² is not a hydrogen atom;

R³ is selected from C₁₋₆ alkyl, C₂₋₆ alkenyl, C₃₋₆ cycloalkyl, C₃₋₆ cycloalkyl-C₁₋₆ alkyl, C₁₋₆ alkoxy, aryl, and aryl-C₁₋₃ alkyl, wherein each aryl moiety is optionally substituted with from one to three substituents independently selected from C₁₋₃ alkyl, C₁₋₃ alkoxy, amino, C₁₋₃ alkylamino, and halogeno; and

wherein each of the aforementioned alkyl, alkenyl, cycloalkyl, and alkoxy moieties are optionally substituted with from one to three fluorine atoms.

One aspect of the invention provides a method of making a compound of Formula 1, above, including a diastereomer thereof, or a pharmaceutically acceptable complex, salt, solvate or hydrate thereof. The method comprises the steps of:

(a) reducing a cyano moiety of a compound of Formula 8, 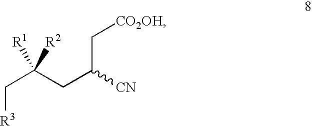
or a salt thereof to give a compound of Formula 9, 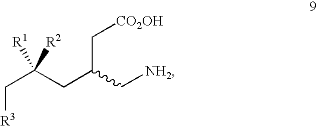
or a salt thereof, wherein R¹, R², and R³ in Formula 8 and Formula 9 are as defined for Formula 1;

(b) optionally treating a salt of the compound of Formula 9 with an acid;

(c) resolving the compound of Formula 9 or a salt thereof; and

(d) optionally converting the compound of Formula 1 or a salt thereof into a pharmaceutically acceptable complex, salt, solvate or hydrate thereof.

Another aspect of the invention provides a method of making a compound of Formula 1, above, a diastereomer thereof, or pharmaceutically acceptable complex, salt, solvate or hydrate thereof. The method comprises the steps of:

(a) reducing a cyano moiety of a compound of Formula 12, 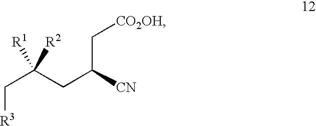
a diastereomer thereof, or a salt thereof, wherein R¹, R², and R³ in Formula 12 are as defined for Formula 1; and

(b) optionally converting the compound of Formula 1 or a salt thereof into a pharmaceutically acceptable complex, salt, solvate or hydrate thereof.

A further aspect of the invention provides a method of making a compound of Formula 12, above, The method comprises the steps of:

(a) contacting a compound of Formula 7, 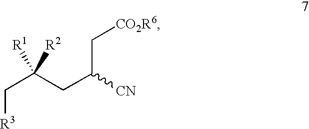
with an enzyme to yield the compound of Formula 10, 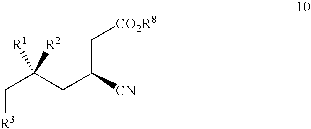
or a salt thereof, and a compound of Formula 11, 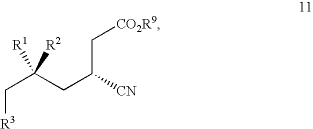
or a salt thereof, wherein the enzyme diastereoselectively hydrolyzes the compound of Formula 7 to the compound of Formula 10 or a salt thereof, or to a compound of Formula 11 or a salt thereof;

(b) isolating the compound of Formula 10, a diastereomer thereof, or a salt thereof, and

(c) optionally hydrolyzing the compound of Formula 10 or a diastereomer thereof, to give the compound of Formula 12, or a diastereomer thereof, wherein

R¹, R², and R³ in Formula 7, Formula 10, and Formula 11 are as defined for Formula 1, above;

R⁶ in Formula 7 is selected from C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl; and

R⁸ and R⁹ in Formula 10 and 11 are each independently selected from hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl;

wherein each of the aforementioned aryl moieties may be optionally substituted with from one to three substituents independently selected from C₁₋₃ alkyl, C₁₋₃ alkoxy, amino, C₁₋₃ alkylamino, and halogeno.

An additional aspect of the invention provides a compound of Formula 19, 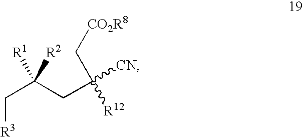
including salts thereof, wherein R¹, R², and R³ are as defined for Formula 1, above;

R⁸ is selected from hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl;

R¹² is a hydrogen atom or —C(O)OR⁷; and

R⁷ is selected from C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl;

wherein each of the aforementioned aryl moieties is optionally substituted with from one to three substituents independently selected from C₁₋₃ alkyl, C₁₋₃ alkoxy, amino, C₁₋₃ alkylamino, and halogeno; and

wherein each of the aforementioned alkyl, alkenyl, cycloalkyl, and alkoxy moieties are optionally substituted with from one to three fluorine atoms.

A further aspect of the invention provides compounds of Formula 7, Formula 8, Formula 10, Formula 11, and Formula 12, above, including their diastereomers, opposite enantiomers, and where possible, their complexes, salts, solvates and hydrates. These compounds include:

(2′R)-2-cyano-2-(2′-methyl-butyl)-succinic acid diethyl ester;

(2′R)-2-cyano-2-(2′-methyl-pentyl)-succinic acid diethyl ester;

(2′R)-2-cyano-2-(2′-methyl-hexyl)-succinic acid diethyl ester;

(2′R)-2-cyano-2-(2′,4′-dimethyl-pentyl)-succinic acid diethyl ester;

(5R)-3-cyano-5-methyl-heptanoic acid ethyl ester;

(5R)-3-cyano-5-methyl-octanoic acid ethyl ester;

(5R)-3-cyano-5-methyl-nonanoic acid ethyl ester;

(5R)-3-cyano-5,7-dimethyl-octanoic acid ethyl ester;

(5R)-3-cyano-5-methyl-heptanoic acid;

(5R)-3-cyano-5-methyl-octanoic acid;

(5R)-3-cyano-5-methyl-nonanoic acid;

(5R)-3-cyano-5,7-dimethyl-octanoic acid;

(3S,5R)-3-cyano-5-methyl-heptanoic acid;

(3S,5R)-3-cyano-5-methyl-octanoic acid;

(3S,5R)-3-cyano-5-methyl-nonanoic acid;

(3S,5R)-3-cyano-5,7-dimethyl-octanoic acid;

(3S,5R)-3-cyano-5-methyl-heptanoic acid ethyl ester;

(3S,5R)-3-cyano-5-methyl-octanoic acid ethyl ester;

(3S,5R)-3-cyano-5-methyl-nonanoic acid ethyl ester;

(3S,5R)-3-cyano-5,7-dimethyl-octanoic acid ethyl ester;

(3R,5R)-3-cyano-5-methyl-heptanoic acid;

(3R,5R)-3-cyano-5-methyl-octanoic acid;

(3R,5R)-3-cyano-5-methyl-nonanoic acid;

(3R,5R)-3-cyano-5,7-dimethyl-octanoic acid;

(3R,5R)-3-cyano-5-methyl-heptanoic acid ethyl ester;

(3R,5R)-3-cyano-5-methyl-octanoic acid ethyl ester;

(3R,5R)-3-cyano-5-methyl-nonanoic acid ethyl ester;

(3R,5R)-3-cyano-5,7-dimethyl-octanoic acid ethyl ester; and

diastereomers and opposite enantiomers of the aforementioned compounds, and salts of the aforementioned compounds, their diastereomers and opposite enantiomers.

The present invention includes all complexes and salts, whether pharmaceutically acceptable or not, solvates, hydrates, and polymorphic forms of the disclosed compounds. Certain compounds may contain an alkenyl or cyclic group, so that cis/trans (or Z/E) stereoisomers are possible, or may contain a keto or oxime group, so that tautomerism may occur. In such cases, the present invention generally includes all Z/E isomers and tautomeric forms, whether they are pure, substantially pure, or mixtures.

### DETAILED DESCRIPTION

Definitions and Abbreviations

Unless otherwise indicated, this disclosure uses definitions provided below. Some of the definitions and formulae may include a dash (“—”) to indicate a bond between atoms or a point of attachment to a named or unnamed atom or group of atoms. Other definitions and formulae may include an equal sign (“═”) or an identity symbol (“≡”) to indicate a double bond or a triple bond, respectively. Certain formulae may also include one or more asterisks (“*”) to indicate stereogenic (asymmetric or chiral) centers, although the absence of an asterisk does not indicate that the compound lacks a stereocenter. Such formulae may refer to the racemate or to individual enantiomers or to individual diastereomers, which may or may not be pure or substantially pure. Other formulae may include one or more wavy bonds (“ [symbol] ”). When attached to a stereogenic center, the wavy bonds refer to both stereoisomers, either individually or as mixtures. Likewise, when attached to a double bond, the wavy bonds indicate a Z-isomer, an E-isomer, or a mixture of Z and E isomers. Some formulae may include a dashed bond “ [symbol] ” to indicate a single or a double bond.

“Substituted” groups are those in which one or more hydrogen atoms have been replaced with one or more non-hydrogen atoms or groups, provided that valence requirements are met and that a chemically stable compound results from the substitution.

“About” or “approximately,” when used in connection with a measurable numerical variable, refers to the indicated value of the variable and to all values of the variable that are within the experimental error of the indicated value (e.g., within the 95% confidence interval for the mean) or within ±10 percent of the indicated value, whichever is greater.

“Alkyl” refers to straight chain and branched saturated hydrocarbon groups, generally having a specified number of carbon atoms (i.e., C₁₋₆ alkyl refers to an alkyl group having 1, 2, 3, 4, 5, or 6 carbon atoms and C₁₋₁₂ alkyl refers to an alkyl group having 1, 2, 3, 4, 5, 6, 7, 8, 9, 10, 11, or 12 carbon atoms). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkenyl” refers to straight chain and branched hydrocarbon groups having one or more unsaturated carbon-carbon bonds, and generally having a specified number of carbon atoms. Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Alkanoyl” refers to alkyl-C(O)—, where alkyl is defined above, and generally includes a specified number of carbon atoms, including the carbonyl carbon. Examples of alkanoyl groups include formyl, acetyl, propionyl, butyryl, pentanoyl, hexanoyl, and the like.

“Alkenoyl” and “alkynoyl” refer, respectively, to alkenyl-C(O)— and alkynyl-C(O)—, where alkenyl and alkynyl are defined above. References to alkenoyl and alkynoyl generally include a specified number of carbon atoms, excluding the carbonyl carbon. Examples of alkenoyl groups include propenoyl, 2-methylpropenoyl, 2-butenoyl, 3-butenoyl, 2-methyl-2-butenoyl, 2-methyl-3-butenoyl, 3-methyl-3-butenoyl, 2-pentenoyl, 3-pentenoyl, 4-pentenoyl, and the like. Examples of alkynoyl groups include propynoyl, 2-butynoyl, 3-butynoyl, 2-pentynoyl, 3-pentynoyl, 4-pentynoyl, and the like.

“Alkoxy” and “alkoxycarbonyl” refer, respectively, to alkyl-O—, alkenyl-O, and alkynyl-O, and to alkyl-O—C(O)—, alkenyl-O—C(O)—, alkynyl-O—C(O)—, where alkyl, alkenyl, and alkynyl are defined above. Examples of alkoxy groups include methoxy, ethoxy, n-propoxy, i-propoxy, n-butoxy, s-butoxy, t-butoxy, n-pentoxy, s-pentoxy, and the like. Examples of alkoxycarbonyl groups include methoxycarbonyl, ethoxycarbonyl, n-propoxycarbonyl, i-propoxycarbonyl, n-butoxycarbonyl, s-butoxycarbonyl, t-butoxycarbonyl, n-pentoxycarbonyl, s-pentoxycarbonyl, and the like.

“Halo,” “halogen” and “halogeno” may be used interchangeably, and refer to fluoro, chloro, bromo, and iodo.

“Haloalkyl,” “haloalkenyl,” “haloalkynyl,” “haloalkanoyl,” “haloalkenoyl,” “haloalkynoyl,” “haloalkoxy,” and “haloalkoxycarbonyl” refer, respectively, to alkyl, alkenyl, alkynyl, alkanoyl, alkenoyl, alkynoyl, alkoxy, and alkoxycarbonyl groups substituted with one or more halogen atoms, where alkyl, alkenyl, alkynyl, alkanoyl, alkenoyl, alkynoyl, alkoxy, and alkoxycarbonyl are defined above. Examples of haloalkyl groups include trifluoromethyl, trichloromethyl, pentafluoroethyl, pentachloroethyl, and the like.

“Cycloalkyl” refers to saturated monocyclic and bicyclic hydrocarbon rings, generally having a specified number of carbon atoms that comprise the ring (i.e., C₃₋₇ cycloalkyl refers to a cycloalkyl group having 3, 4, 5, 6 or 7 carbon atoms as ring members). The cycloalkyl may be attached to a parent group or to a substrate at any ring atom, unless such attachment would violate valence requirements. Likewise, the cycloalkyl groups may include one or more non-hydrogen substituents unless such substitution would violate valence requirements. Useful substituents include alkyl, alkenyl, alkynyl, haloalkyl, haloalkenyl, haloalkynyl, alkoxy, alkoxycarbonyl, alkanoyl, and halo, as defined above, and hydroxy, mercapto, nitro, and amino.

Examples of monocyclic cycloalkyl groups include cyclopropyl, cyclobutyl, cyclopentyl, cyclohexyl, and the like. Examples of bicyclic cycloalkyl groups include bicyclo[1.1.0]butyl, bicyclo[1.1.1]pentyl, bicyclo[2.1.1]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like.

“Cycloalkenyl” refers monocyclic and bicyclic hydrocarbon rings having one or more unsaturated carbon-carbon bonds and generally having a specified number of carbon atoms that comprise the ring (i.e., C₃₋₇ cycloalkenyl refers to a cycloalkenyl group having 3, 4, 5, 6 or 7 carbon atoms as ring members). The cycloalkenyl may be attached to a parent group or to a substrate at any ring atom, unless such attachment would violate valence requirements. Likewise, the cycloalkenyl groups may include one or more non-hydrogen substituents unless such substitution would violate valence requirements. Useful substituents include alkyl, alkenyl, alkynyl, haloalkyl, haloalkenyl, haloalkynyl, alkoxy, alkoxycarbonyl, alkanoyl, and halo, as defined above, and hydroxy, mercapto, nitro, and amino.

“Cycloalkanoyl” and “cycloalkenoyl” refer to cycloalkyl-C(O)— and cycloalkenyl-C(O)—, respectively, where cycloalkyl and cycloalkenyl are defined above. References to cycloalkanoyl and cycloalkenoyl generally include a specified number of carbon atoms, excluding the carbonyl carbon. Examples of cycloalkanoyl groups include cyclopropanoyl, cyclobutanoyl, cyclopentanoyl, cyclohexanoyl, cycloheptanoyl, 1-cyclobutenoyl, 2-cyclobutenoyl, 1-cyclopentenoyl, 2-cyclopentenoyl, 3-cyclopentenoyl, 1-cyclohexenoyl, 2-cyclohexenoyl, 3-cyclohexenoyl, and the like.

“Cycloalkoxy” and “cycloalkoxycarbonyl” refer, respectively, to cycloalkyl-O— and cycloalkenyl-O and to cycloalkyl-O—C(O)— and cycloalkenyl-O—C(O)—, where cycloalkyl and cycloalkenyl are defined above. References to cycloalkoxy and cycloalkoxycarbonyl generally include a specified number of carbon atoms, excluding the carbonyl carbon. Examples of cycloalkoxy groups include cyclopropoxy, cyclobutoxy, cyclopentoxy, cyclohexoxy, 1-cyclobutenoxy, 2-cyclobutenoxy, 1-cyclopentenoxy, 2-cyclopentenoxy, 3-cyclopentenoxy, 1-cyclohexenoxy, 2-cyclohexenoxy, 3-cyclohexenoxy, and the like. Examples of cycloalkoxycarbonyl groups include cyclopropoxycarbonyl, cyclobutoxycarbonyl, cyclopentoxycarbonyl, cyclohexoxycarbonyl, 1-cyclobutenoxycarbonyl, 2-cyclobutenoxycarbonyl, 1-cyclopentenoxycarbonyl, 2-cyclopentenoxycarbonyl, 3-cyclopentenoxycarbonyl, 1-cyclohexenoxycarbonyl, 2-cyclohexenoxycarbonyl, 3-cyclohexenoxycarbonyl, and the like.

“Aryl” and “arylene” refer to monovalent and divalent aromatic groups, respectively, including 5- and 6-membered monocyclic aromatic groups that contain 0 to 4 heteroatoms independently selected from nitrogen, oxygen, and sulfur. Examples of monocyclic aryl groups include phenyl, pyrrolyl, furanyl, thiopheneyl, thiazolyl, isothiazolyl, imidazolyl, triazolyl, tetrazolyl, pyrazolyl, oxazolyl, isooxazolyl, pyridinyl, pyrazinyl, pyridazinyl, pyrimidinyl, and the like. Aryl and arylene groups also include bicyclic groups, tricyclic groups, etc., including fused 5- and 6-membered rings described above. Examples of multicyclic aryl groups include naphthyl, biphenyl, anthracenyl, pyrenyl, carbazolyl, benzoxazolyl, benzodioxazolyl, benzothiazolyl, benzoimidazolyl, benzothiopheneyl, quinolinyl, isoquinolinyl, indolyl, benzofuranyl, purinyl, indolizinyl, and the like. They aryl and arylene groups may be attached to a parent group or to a substrate at any ring atom, unless such attachment would violate valence requirements. Likewise, aryl and arylene groups may include one or more non-hydrogen substituents unless such substitution would violate valence requirements. Useful substituents include alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino.

“Heterocycle” and “heterocyclyl” refer to saturated, partially unsaturated, or unsaturated monocyclic or bicyclic rings having from 5 to 7 or from 7 to 11 ring members, respectively. These groups have ring members made up of carbon atoms and from 1 to 4 heteroatoms that are independently nitrogen, oxygen or sulfur, and may include any bicyclic group in which any of the above-defined monocyclic heterocycles are fused to a benzene ring. The nitrogen and sulfur heteroatoms may optionally be oxidized. The heterocyclic ring may be attached to a parent group or to a substrate at any heteroatom or carbon atom unless such attachment would violate valence requirements. Likewise, any of the carbon or nitrogen ring members may include a non-hydrogen substituent unless such substitution would violate valence requirements. Useful substituents include alkyl, alkenyl, alkynyl, haloalkyl, haloalkenyl, haloalkynyl, cycloalkyl, cycloalkenyl, alkoxy, cycloalkoxy, alkanoyl, cycloalkanoyl, cycloalkenoyl, alkoxycarbonyl, cycloalkoxycarbonyl, and halo, as defined above, and hydroxy, mercapto, nitro, amino, and alkylamino.

Examples of heterocycles include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Heteroaryl” and “heteroarylene” refer, respectively, to monovalent and divalent heterocycles or heterocyclyl groups, as defined above, which are aromatic. Heteroaryl and heteroarylene groups represent a subset of aryl and arylene groups, respectively.

“Arylalkyl” and “heteroarylalkyl” refer, respectively, to aryl-alkyl and heteroaryl-alkyl, where aryl, heteroaryl, and alkyl are defined above. Examples include benzyl, fluorenylmethyl, imidazol-2-yl-methyl, and the like.

“heteroarylalkanoyl,” “arylalkenoyl,” “heteroarylalkenoyl,” “arylalkynoyl,” and “heteroarylalkynoyl” refer, respectively, to aryl-alkanoyl, heteroaryl-alkanoyl, aryl-alkenoyl, heteroaryl-alkenoyl, aryl-alkynoyl, and heteroaryl-alkynoyl, where aryl, heteroaryl, alkanoyl, alkenoyl, and alkynoyl are defined above. Examples include benzoyl, benzylcarbonyl, fluorenoyl, fluorenylmethylcarbonyl, imidazol-2-oyl, imidazol-2-yl-methylcarbonyl, phenylethenecarbonyl, 1-phenylethenecarbonyl, 1-phenyl-propenecarbonyl, 2-phenyl-propenecarbonyl, 3-phenyl-propenecarbonyl, imidazol-2-yl-ethenecarbonyl, 1-(imidazol-2-yl)-ethenecarbonyl, 1-(imidazol-2-yl)-propenecarbonyl, 2-(imidazol-2-yl)-propenecarbonyl, 3-(imidazol-2-yl)-propenecarbonyl, phenylethynecarbonyl, phenylpropynecarbonyl, (imidazol-2-yl)-ethynecarbonyl, (imidazol-2-yl)-propynecarbonyl, and the like.

“Arylalkoxy” and “heteroarylalkoxy” refer, respectively, to aryl-alkoxy and heteroaryl-alkoxy, where aryl, heteroaryl, and alkoxy are defined above. Examples include benzyloxy, fluorenylmethyloxy, imidazol-2-yl-methyloxy, and the like.

“Aryloxy” and “heteroaryloxy” refer, respectively, to aryl-O— and heteroaryl-O—, where aryl and heteroaryl are defined above. Examples include phenoxy, imidazol-2-yloxy, and the like.

“Aryloxycarbonyl,” “heteroaryloxycarbonyl,” “arylalkoxycarbonyl,” and “heteroarylalkoxycarbonyl” refer, respectively, to aryloxy-C(O)—, heteroaryloxy-C(O)—, arylalkoxy-C(O)—, and heteroarylalkoxy-C(O)—, where aryloxy, heteroaryloxy, arylalkoxy, and heteroarylalkoxy are defined above. Examples include phenoxycarbonyl, imidazol-2-yloxycarbonyl, benzyloxycarbonyl, fluorenylmethyloxycarbonyl, imidazol-2-yl-methyloxycarbonyl, and the like.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate), and arylsulfonates (e.g., tosylate, brosylate, closylate, and nosylate). Others include carbonates, halide ions, carboxylate anions, phenolate ions, and alkoxides. Some stronger bases, such as NH₂⁻ and OH⁻ can be made better leaving groups by treatment with an acid. Common electrofugal leaving groups include the proton, CO₂, and metals.

“Enantiomeric excess” or “ee” is a measure, for a given sample, of the excess of one enantiomer over a racemic sample of a chiral compound and is expressed as a percentage. Enantiomeric excess is defined as 100×(er−1)/(er+1), where “er” is the ratio of the more abundant enantiomer to the less abundant enantiomer.

“Diastereomeric excess” or “de” is a measure, for a given sample, of the excess of one diastereomer over a sample having equal amounts of diastereomers and is expressed as a percentage. Diastereomeric excess is defined as 100×(dr−1)/(dr+1), where “dr” is the ratio of a more abundant diastereomer to a less abundant diastereomer.

“Stereoselective,” “enantioselective,” “diastereoselective,” and variants thereof, refer to a given process (e.g., hydrogenation) that yields more of one stereoisomer, enantiomer, or diastereoisomer than of another, respectively.

“High level of stereoselectivity,” “high level of enantioselectivity,” “high level of diastereoselectivity,” and variants thereof, refer to a given process that yields products having an excess of one stereoisomer, enantiomer, or diastereoisomer, which comprises at least about 90% of the products. For a pair of enantiomers or diastereomers, a high level of enantioselectivity or diastereoselectivity would correspond to an ee or de of at least about 80%.

“Stereoisomerically enriched,” “enantiomerically enriched,” “diastereomerically enriched,” and variants thereof, refer, respectively, to a sample of a compound that has more of one stereoisomer, enantiomer or diastereomer than another. The degree of enrichment may be measured by % of total product, or for a pair of enantiomers or diastereomers, by ee or de.

“Substantially pure stereoisomer,” “substantially pure enantiomer,” “substantially pure diastereomer,” and variants thereof, refer, respectively, to a sample containing a stereoisomer, enantiomer, or diastereomer, which comprises at least about 95% of the sample. For pairs of enantiomers and diastereomers, a substantially pure enantiomer or diastereomer would correspond to samples having an ee or de of about 90% or greater.

A “pure stereoisomer,” “pure enantiomer,” “pure diastereomer,” and variants thereof, refer, respectively, to a sample containing a stereoisomer, enantiomer, or diastereomer, which comprises at least about 99.5% of the sample. For pairs of enantiomers and diastereomers, a pure enantiomer or pure diastereomer” would correspond to samples having an ee or de of about 99% or greater.

“Opposite enantiomer” refers to a molecule that is a non-superimposable mirror image of a reference molecule, which may be obtained by inverting all of the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomers” of a specified compound refer to the opposite enantiomer of the compound and to any diastereoisomers or geometric isomers (Z/E) of the compound. For example, if the specified compound has S,R,Z stereochemical configuration, its stereoisomers would include its opposite enantiomer having R,S,Z configuration, its diastereomers having S,S,Z configuration and R,R,Z configuration, and its geometric isomers having S,R,E configuration, R,S,E configuration, S,S,E configuration, and R,R,E configuration.

“Enantioselectivity value” or “E” refers to the ratio of specificity constants for each enantiomer (or for each stereoisomer of a pair of diastereomers) of a compound undergoing chemical reaction or conversion and may be calculated (for the S-enantiomer) from the expression,

$$\begin{matrix}E = \frac{{K}_{S} / {K}_{SM}}{{K}_{R} / {K}_{RM}} \\ = \frac{ln ⌊ 1 - χ ( 1 + {ee}_{p} ) ⌋}{ln [ 1 - χ ( 1 - {ee}_{p} ) ]} \\ = \frac{ ln [ 1 - χ ( 1 - {ee}_{S} ) ]}{ln [ 1 - χ ( 1 + {ee}_{S} ) ]} ,\end{matrix}$$

where K_{S} and K_{R} are the 1st order rate constants for the conversion of the S- and R-enantiomers, respectively; K_{SM} and K_{RM} are the Michaelis constants for the S- and R-enantiomers, respectively; χ is the fractional conversion of the substrate; eeₚ and eeₛ are the enantiomeric excess of the product and substrate (reactant), respectively.

“Lipase Unit” or “LU” refers to the amount of enzyme (in g) that liberates 1 μmol of titratable butyric acid/min when contacted with tributyrin and an emulsifier (gum arabic) at 30° C. and pH 7.

“Solvate” refers to a molecular complex comprising a disclosed or claimed compound and a stoichiometric or non-stoichiometric amount of one or more solvent molecules (e.g., EtOH).

“Hydrate” refers to a solvate comprising a disclosed or claimed compound and a stoichiometric or non-stoichiometric amount of water.

“Pharmaceutically acceptable complexes, salts, solvates, or hydrates” refers to complexes, acid or base addition salts, solvates or hydrates of claimed and disclosed compounds, which are within the scope of sound medical judgment, suitable for use in contact with the tissues of patients without undue toxicity, irritation, allergic response, and the like, commensurate with a reasonable benefit/risk ratio, and effective for their intended use.

“Pre-catalyst” or “catalyst precursor” refers to a compound or set of compounds that are converted into a catalyst prior to use.

“Treating” refers to reversing, alleviating, inhibiting the progress of, or preventing a disorder or condition to which such term applies, or to preventing one or more symptoms of such disorder or condition.

“Treatment” refers to the act of “treating,” as defined immediately above.

Table 1 lists abbreviations used throughout the specification.

TABLE 1
List of Abbreviations

| Abbreviation | Description |
|---|---|
| Ac | acetyl |
| ACN | acetonitrile |
| Ac₂O | acetic anhydride |
| aq | aqueous |
| (R,R)-BDPP | (2R,4R)-(+)-2,4-bis(diphenylphosphino)pentane |
| BES | N,N-bis(2-hydroxyethyl)-2-aminoethanesulfonic acid |
| (R)-BICHEP | (R)-(−)-2,2′-bis(dicyclohexylphosphino)-6,6′-dimethyl-1,1′- |
| | biphenyl |
| BICINE | N,N-bis(2-hydroxyethyl)glycine |
| (S,S)-BICP | (2S,2′S)-bis(diphenylphosphino)-(1S,1′S)-bicyclopentane |
| BIFUP | 2,2′-bis(diphenylphosphino)-4,4′,6,6′- |
| | tetrakis(trifluoromethyl)-1,1′-biphenyl |
| (R)-Tol-BINAP | (R)-(+)-2,2′-bis(di-p-tolylphosphino)-1,1′-binaphthyl |
| (S)-Tol-BINAP | (S)-(+)-2,2′-bis(di-p-tolylphosphino)-1,1′-binaphthyl |
| (R)-BINAP | (R)-2,2′-bis(diphenylphosphino)-1′1-binaphthyl |
| (S)-BINAP | (S)-2,2′-bis(diphenylphosphino)-1′1-binaphthyl |
| BIPHEP | 2,2′-bis(diphenylphosphino)-1,1′-biphenyl |
| (R)—MeO-BIPHEP | (R)-(6,6′-dimethoxybiphenyl-2,2′-diyl)- |
| | bis(diphenylphosphine) |
| (R)—Cl—MeO-BIPHEP | (R)-(+)-5,5′-dichloro-6,6′-dimethoxy-2,2′- |
| | bis(diphenylphosphino)-1,1′-biphenyl |
| (S)—Cl—MeO-BIPHEP | (S)-(+)-5,5′-dichloro-6,6′-dimethoxy-2,2′- |
| | bis(diphenylphosphino)-1,1′-biphenyl |
| BisP* | (S,S)-1,2-bis(t-butylmethylphosphino)ethane |
| (+)-tetraMeBITIANP | (S)-(+)-2,2′-bis(diphenylphosphino)-4,4′,6,6′-tetramethyl- |
| | 3,3′-bibenzo[b]thiophene |
| Bn | benzyl |
| BnBr, BnCl | benzylbromide, benzylchloride |
| Boc | t-butoxycarbonyl |
| BOP | benzotriazol-1-yloxy-tris-(dimethylamino)-phosphonium |
| | hexafluorophosphate |
| (R)—(S)-BPPFA | (−)-(R)--N,N-dimethyl-1-((S)-1′,2- |
| | bis(diphenylphosphino)ferrocenyl)ethylamine |
| (R,R)—Et-BPE | (+)-1,2-bis((2R,5i)-2,5-diethylphospholano)ethane |
| (R,R)—Me-BPE | (+)-1,2-bis((2R,5R)-2,5-dimethylphospholano)ethane |
| (S,S)-BPPM | (−)-(2S,4S)-2-diphenylphosphinomethyl-4- |
| | diphenylphosphino-1-t-butoxycarbonylpyrrolidine |
| Bs | brosyl or p-bromo-benzenesulfonyl |
| Bu | butyl |
| n-BuLi | n-butyl lithium |
| t-Bu | tertiary butyl |
| Bu₄N⁺Br⁻ | tetrabutyl-ammonium bromide |
| t-BuOK | potassium tertiary-butoxide |
| t-BuOLi | lithium tertiary-butoxide |
| t-BuOMe | tertiary butyl methyl ether |
| t-BuONa | sodium tertiary butyl oxide |
| (+)-CAMP | (R)-(+)-cyclohexyl(2-anisyl)methylphosphine; a |
| | monophosphine |
| CARBOPHOS | methyl-α-D-glucopyranoside-2,6-dibenzoate-3,4-di(bis(3,5- |
| | dimethylphenyl)phosphinite) |
| Cbz | benzyloxycarbonyl |
| CDI | N,N-carbonyldiimidazole |
| χ | fractional conversion |
| CnTunaPHOS | 2,2′-bis-diphenylphosphanyl-biphenyl having an —O— |
| | (CH₂)ₙ—O—group linking the 6,6′ carbon atoms of the |
| | biphenyl (e.g., (R)-1,14-bis-diphenylphosphanyl-6,7,8,9- |
| | tetrahydro-5,10-dioxa-dibenzo[a,c]cyclodecene for n = 4). |
| COD | 1,5-cyclooctadiene |
| (R)-CYCPHOS | (R)-1,2-bis(diphenylphosphino)-1-cyclohexylethane |
| DABCO | 1,4-diazabicyclo[2.2.2]octane |
| DBAD | di-t-butyl azodicarboxylate |
| DBN | 1,5-diazabicyclo[4.3.0]non-5-ene |
| DBU | 1,8-diazabicyclo[5.4.0]undec-7-ene |
| DCC | dicycohexylcarbodiimide |
| de | diastereomeric excess |
| DEAD | diethyl azodicarboxylate |
| (R,R)-DEGUPHOS | N-benzyl-(3R,4R)-3,4-bis(diphenylphosphino)pyrrolidine |
| DIAD | diisopropyl azodicarboxylate |
| (R,R)-DIOP | (4R,5R)-(−)-O-isopropylidene-2,3-dihydroxy-1,4- |
| | bis(diphenylphosphino)butane |
| (R,R)-DIPAMP | (R,R)-(−)-1,2-bis[(O- |
| | methoxyphenyl)(phenyl)phosphino]ethane |
| DIPEA | diisopropylethylamine (Hunig's Base) |
| DMAP | 4-(dimethylamino) pyridine |
| DMF | dimethylformamide |
| DMSO | dimethylsulfoxide |
| DMT-MM | 4-(4,6-dimethoxy-1,3,5-triazin-2-yl)-4-methylmorpholinium |
| | chloride |
| (R,R)—Et-DUPHOS | (−)-1,2-bis((2R,5R)-2,5-diethylphospholano)benzene |
| (S,S)—Et-DUPHOS | (−)-1,2-bis((2S,5S)-2,5-diethylphospholano)benzene |
| (R,R)-i-Pr-DUPHOS | (+)-1,2-bis((2R,5R)-2,5-di-i-propylphospholano)benzene |
| (R,R)—Me-DUPHOS | (−)-1,2-bis((2R,5R)-2,5-dimethylphospholano)benzene |
| (S,S)—Me-DUPHOS | (−)-1,2-bis((2S,5S)-2,5-dimethylphospholano)benzene |
| E | Enantioselectivity value or ratio of specificity constants for |
| | each enantiomer of a compound undergoing chemical |
| | reaction or conversion |
| EDCI | 1-(3-dimethylaminopropyl)-3-ethylcarbodiimide |
| ee (eeₚ or eeₛ) | enantiomeric excess (of product or reactant) |
| eq | equivalents |
| er | enantiomeric ratio |
| Et | ethyl |
| Et₃N | triethyl-amine |
| EtOAc | ethyl acetate |
| Et₂O | diethyl ether |
| EtOH | ethyl alcohol |
| FDPP | pentafluorophenyl diphenylphosphinate |
| (R,R)—Et-FerroTANE | 1,1′-bis((2R,4R)-2,4-diethylphosphotano)ferrocene |
| Fmoc | 9-fluoroenylmethoxycarbonyl |
| GC | gas chromatography |
| h, min, s | hour(s), minute(s), second(s) |
| HEPES | 4-(2-hydroxyethyl)piperazine-1-ethanesulfonic acid |
| HOAc | acetic acid |
| HOAt | 1-hydroxy-7-azabenzotriazole |
| HOBt | N-hydroxybenzotriazole |
| HODhbt | 3-hydroxy-3,4-dihydro-4-oxo-1,2,3-benzotriazine |
| HPLC | high performance liquid chromatography |
| IAcOEt | ethyl iodoacetate |
| IPA | isopropanol |
| i-PrOAc | isopropyl acetate |
| (R)—(R)-JOSIPHOS | (R)-(−)-1-[(R)-2- |
| | (diphenylphosphino)ferrocenyl]ethyldicyclohexylphosphine |
| (S)—(S)-JOSIPHOS | (S)-(−)-1-[(S)-2- |
| | (diphenylphosphino)ferrocenyl]ethyldicyclohexylphosphine |
| (R)—(S)-JOSIPHOS | (R)-(−)-1-[(S)-2- |
| | (diphenylphosphino)ferrocenyl]ethyldicyclohexylphosphine |
| KHMDS | potassium hexamethyldisilazane |
| KF | Karl Fischer |
| K_{S}, K_{S} | 1st order rate constant for S- or R-enantiomer |
| K_{SM}, K_{RM} | Michaelis constant for S- or R-enantiomer |
| LAH | lithium aluminum hydride |
| LC/MS | liquid chromatography mass spectrometry |
| LDA | lithium diisopropylamide |
| LHMDS | lithium hexamethyldisilazane |
| LICA | lithium isopropylcyclohexylamide |
| LTMP | 2,2,6,6-tetramethylpiperidine |
| LU | lipase unit |
| Me | methyl |
| MeCl₂ | methylene chloride |
| MeI | methyl iodide |
| MEK | methylethylketone or butan-2-one |
| MeOH | methyl alcohol |
| MeONa | sodium methoxide |
| MES | 2-morpholinoethanesulfonic acid |
| (R,R)-t-butyl-miniPHOS | (R,R)-1,2-bis(di-t-butylmethylphosphino)methane |
| (S,S) MandyPhos | (S,S)-(−)-2,2′-bis[(R)-(N,N-dimethylamino) (phenyl)methyl]- |
| | 1,1′-bis(diphenylphosphino)ferrocene |
| (R)-MonoPhos | (R)-(−)-[4,N,N-dimethylamino]dinaphtho[2,1-d:1′,2′- |
| | f][1,3,2]dioxaphosphepin |
| (R)-MOP | (R)-(+)-2-(diphenylphosphino)-2′-methoxy-1,1′-binaphthyl |
| MOPS | 3-(N-morpholino)propanesulfonic acid |
| MPa | mega Pascals |
| mp | melting point |
| Ms | mesyl or methanesulfonyl |
| MTBE | methyl tertiary butyl ether |
| NMP | N-methylpyrrolidone |
| Ns | nosyl or nitrobenzene sulfonyl |
| (R,R)-NORPHOS | (2R,3R)-(−)-2,3-bis(diphenylphosphino)bicyclo[2.2.1]hept-5- |
| | ene |
| OTf⁻ | triflate (trifluoro-methanesulfonic acid anion) |
| PdCl₂(dppf)₂ | dichloro[1,1′-bis(diphenylphosphino)ferrocene]palladium |
| | (II) dichloromethane adduct |
| (R,S,R,S)—Me— | (1R,2S,4R,5S)-2,5-dimethyl-7-phosphadicyclo[2.2.1]heptane |
| PENNPHOS | |
| Ph | phenyl |
| Ph₃P | triphenylphosphine |
| Ph₃As | triphenylarsine |
| (R)-PHANEPHOS | (R)-(−)-4,12-bis(diphenylphosphino)-[2.2]-paracyclophane |
| (S)-PHANEPHOS | (S)-(−)-4,12-bis(diphenylphosphino)-[2.2]-paracyclophane |
| (R)-PNNP | N,N′-bis[(R)-(+)-α-methylbenzyl]-N,N′- |
| | bis(diphenylphosphino)ethylene diamine |
| PPh₂-PhOx-Ph | (R)-(−)-2-[2-(diphenylphosphino)phenyl]-4-phenyl-2- |
| | oxazoline |
| PIPES | piperazine-1,4-bis(2-ethanesulfonic acid) |
| Pr | propyl |
| i-Pr | isopropyl |
| (R)-PROPHOS | (R)-(+)-1,2-bis(diphenylphosphino)propane |
| PyBOP | benzotriazole-1-yl-oxy-tris-pyrrolidino-phosphonium |
| | hexafluorophosphate |
| (R)-QUINAP | (R)-(+)-1-(2-diphenylphosphino-1-naphthyl)isoquinoline |
| RaNi | Raney nickel |
| RI | refractive index |
| RT | room temperature (approximately 20° C. to 25° C.) |
| s/c | substrate-to-catalyst molar ratio |
| sp | species |
| (R)-SpirOP | (1R,5R,6R)-spiro[4.4]nonane-1,6-diyl-diphenylphosphinous |
| | acid ester; a spirocyclic phosphinite ligand |
| (R,R,S,S) TangPhos | (R,R,S,S) 1,1′-di-t-butyl-[2,2′]biphospholanyl |
| TAPS | N-[tris(hydroxymethyl)methyl]-3-aminopropanesulfonic |
| | acid |
| TATU | O-(7-azabenzotriazol-1-yl)-1,1,3,3-tetramethyluronium |
| | tetrafluoroborate |
| (R)-eTCFP | (R)-2-{[(di-t-butyl-phosphanyl)-ethyl]-methyl-phosphanyl}- |
| | 2-methyl-propane |
| (S)-eTCFP | (S)-2-{[(di-t-butyl-phosphanyl)-ethyl]-methyl-phosphanyl}- |
| | 2-methyl-propane |
| (R)-mTCFP | (R)-2-{[(di-t-butyl-phosphanyl)-methyl]-methyl- |
| | phosphanyl}-2-methyl-propane |
| (S)-mTCFP | (S)-2-{[(di-t-butyl-phosphanyl)-methyl]-methyl- |
| | phosphanyl}-2-methyl-propane |
| TEA | triethanolamine |
| TES | N-[tris(hydroxymethyl)methyl]-2-aminoethanesulfonic acid |
| Tf | triflyl or trifluoromethylsulfonyl |
| TFA | trifluoroacetic acid |
| THF | tetrahydrofuran |
| TLC | thin-layer chromatography |
| TMEDA | N,N,N′,N′-tetramethyl-1,2-ethylenediamine |
| TMS | trimethylsilyl |
| Tr | trityl or triphenylmethyl |
| TRICINE | N-[tris(hydroxymethyl)methyl]glycine |
| Tris buffer | tris(hydroxymethyl)aminomethane buffer |
| TRITON B | benzyltrimethylammonium hydroxide |
| TRIZMA ® | 2-amino-2-(hydroxymethyl)-1,3-propanediol |
| Ts | tosyl or p-toluenesulfonyl |
| p-TSA | para-toluene sulfonic acid |
| v/v | volume percent |
| w/w | weight (mass) percent |

Some of the schemes and examples below may omit details of common reactions, including oxidations, reductions, and so on, separation techniques, and analytical procedures, which are known to persons of ordinary skill in the art of organic chemistry. The details of such reactions and techniques can be found in a number of treatises, including Richard Larock, Comprehensive Organic Transformations (1999), and the multi-volume series edited by Michael B. Smith and others, Compendium of Organic Synthetic Methods (1974-2005). In many cases, starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a diacid, etc.). In addition, in some instances, reaction intermediates may be used in subsequent steps without isolation or purification (i.e., in situ).

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and the like, see T. W. Greene and P. G. Wuts, Protecting Groups in Organic Chemistry (1999) and P. Kocienski, Protective Groups (2000), which are herein incorporated by reference in their entirety for all purposes.

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about RT and ambient pressure, but depending on reaction kinetics, yields, and the like, some reactions may be run at elevated pressures or employ higher (e.g., reflux conditions) or lower (e.g., −70° C. to 0° C.) temperatures. Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Any reference in the disclosure to a stoichiometric range, a temperature range, a pH range, etc., whether or not expressly using the word “range,” also includes the indicated endpoints.

Generally, and unless stated otherwise, when a particular substituent identifier (R¹, R², R³, etc.) is defined for the first time in connection with a formula, the same substituent identifier, when used in a subsequent formula, will have the same definition as in the earlier formula. Thus, for example, if R³⁰ in a first formula is hydrogen atom, halogeno, or C₁₋₆ alkyl, then unless stated differently or otherwise clear from the context of the text, R³⁰ in a second formula is also hydrogen, halogeno, or C₁₋₆ alkyl.

This disclosure concerns materials and methods for preparing optically active γ-amino acids of Formula 1, above, as well as their stereoisomers (e.g., diastereomers and opposite enantiomers) and their pharmaceutically acceptable complexes, salts, solvates and hydrates. The claimed and disclosed methods provide compounds of Formula 1 (or their stereoisomers) that are stereoisomerically enriched, and which in many cases, are pure or substantially pure stereoisomers. For clarity, the specification describes methods and materials for preparing intermediates and final products having specific stereochemical configurations. However, by using starting materials, resolving agents, chiral catalysts, enzymes, and the like, having different stereochemical configurations, the methods may be used to prepare the corresponding diastereomers and opposite enantiomers of the disclosed products and intermediates.

The compounds of Formula 1 have at least two stereogenic centers, as denoted by wedged bonds, and include substituents R¹, R², and R³, which are defined above. Compounds of Formula 1 include those in which R¹ and R² are each independently hydrogen or methyl, provided that R¹ and R² are not both hydrogen, and those in which R³ is C₁₋₆ alkyl, including methyl, ethyl, n-propyl or i-propyl. Representative compounds of Formula 1 also include those in which R¹ is hydrogen, R² is methyl, and R³ is methyl, ethyl, n-propyl, or i-propyl, i.e., (3S,5R)-3-aminomethyl-5-methyl-heptanoic acid, (3S,5R)-3-aminomethyl-5-methyl-octanoic acid, (3S,5R)-3-aminomethyl-5-methyl-nonanoic acid, or (3S,5R)-3-aminomethyl-5,7-dimethyl-octanoic acid. Representative diastereomers of the latter compounds are (3R,5R)- or (3S,5S)-3-aminomethyl-5-methyl-heptanoic acid, (3R,5R) or (3S,5S)-3-aminomethyl-5-methyl-octanoic acid, (3R,5R) or (3S,5S)-3-aminomethyl-5-methyl-nonanoic acid, and (3R,5R) or (3S,5S)-3-aminomethyl-5,7-dimethyl-octanoic acid; representative opposite enantiomers are (3R,5S)-3-aminomethyl-5-methyl-heptanoic acid, (3R,5S)-3-aminomethyl-5-methyl-octanoic acid, (3R,5S)-3-aminomethyl-5-methyl-nonanoic acid, and (3R,5S)-3-aminomethyl-5,7-dimethyl-octanoic acid.

Scheme I shows two methods for preparing compounds of Formula 1. The methods include reacting a chiral alcohol (Formula 2) with an activating agent (Formula 3). The resulting activated alcohol (Formula 4) is reacted with a 2-cyano succinic acid diester (Formula 5) to provide a 2-alkyl-2-cyano succinic acid diester (Formula 6) having a second stereogenic center, which is represented by wavy bonds. The ester moiety that is directly attached to the second asymmetric carbon atom (see Formula 6) is subsequently cleaved to give a 3-cyano carboxylic acid ester (Formula 7), which is converted to the desired product (Formula 1) through contact with either a resolving agent or an enzyme. In the former method, the ester (Formula 7) is hydrolyzed to give a 3-cyano carboxylic acid (Formula 8) or salt. Reduction of the cyano moiety (see Formula 8) gives, upon acidification (if necessary), a γ-amino acid (Formula 9) which is resolved via contact with a resolving agent (e.g., a chiral acid), followed by separation of the desired diastereomeric salt or free amino acid (Formula 1). Alternatively, one diastereomer of the monoester (Formula 7) is diastereoselectively hydrolyzed through contact with an enzyme, which results in a mixture enriched in a 3-cyano carboxylic acid or ester having the requisite stereochemical configuration at C-3 (Formula 10). The ester or acid (Formula 10) is separated from the undesirable diastereomer (Formula 11) and is hydrolyzed (if necessary) to give a pure, or substantially pure, diastereomer of 3-cyano carboxylic acid (Formula 12). Reduction of the cyano moiety gives, upon acid workup (if necessary), the compound of Formula 1.

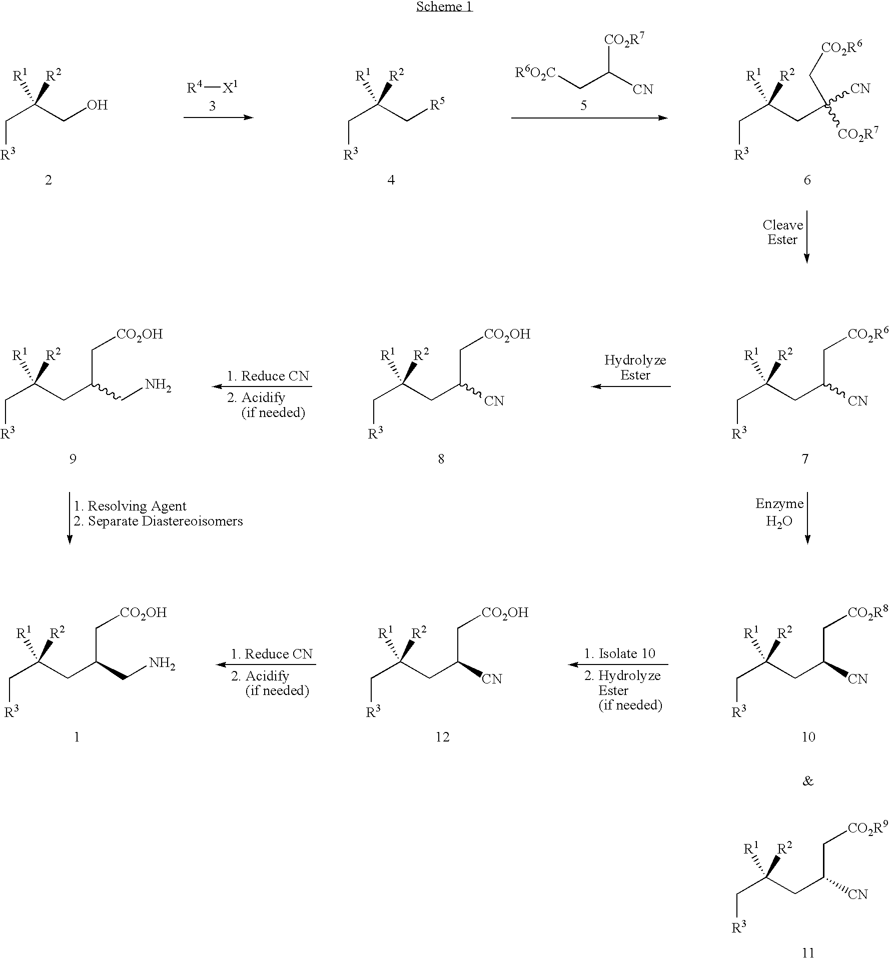

Substituents R¹, R², and R³ in Formula 2, 4, and 6-12 are as defined for Formula 1, above; substituent R⁴ in Formula 3 is selected from tosyl, mesyl, brosyl, closyl (p-chloro-benzenesulfonyl), nosyl, and triflyl; substituent R⁵ in Formula 4 is a leaving group (e.g., R⁴O—); and substituent X¹ in Formula 3 is halogeno (e.g., Cl) or R⁴O—. Substituents R⁶ and R⁷ in Formula 5-7 are each independently selected from C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl. Substituents R⁸ and R⁹ in Formula 10 and 11 are each independently selected from hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl. Each of the aforementioned aryl moieties may be optionally substituted with from one to three substituents independently selected from C₁₋₃ alkyl, C₁₋₃ alkoxy, amino, C₁₋₃ alkylamino, and halogeno.

The chiral alcohol (Formula 2) shown in Scheme I has a stereogenic center at C-2, as denoted by wedge bonds, and includes substituents R¹, R², and R³, which are as defined above. Compounds of Formula 2 include those in which R¹ and R² are each independently hydrogen or methyl, provided that R¹ and R² are not both hydrogen, and those in which R³ is C₁₋₆ alkyl, including methyl, ethyl, n-propyl or i-propyl. Representative compounds of Formula 2 also include those in which R¹ is hydrogen, R² is methyl, and R³ is methyl, ethyl, n-propyl, or i-propyl, i.e., (R)-2-methyl-butan-1-ol, (R)-2-methyl-pentan-1-ol, (R)-2-methyl-hexan-1-ol, or (R)-2,4-dimethyl-pentan-1-ol. Representative opposite enantiomers of the latter compounds are (S)-2-methyl-butan-1-ol, (S)-2-methyl-pentan-1-ol, (S)-2-methyl-hexan-1-ol, and (S)-2,4-dimethyl-pentan-1-ol.

As shown in Scheme I, the hydroxy moiety of the chiral alcohol (Formula 2) is activated via reaction with a compound of Formula 3. The reaction is typically carried out with excess (e.g., about 1.05 eq to about 1.1 eq) activating agent (Formula 3) at a temperature of about −25° C. to about RT. Useful activating agents include sulfonylating agents, such as TsCl, MsCl, BsCl, NsCl, TfCl, and the like, and their corresponding anhydrides (e.g., p-toluenesulfonic acid anhydride). Thus, for example, compounds of Formula 2 may be reacted with TsCl in the presence of pyridine and an aprotic solvent, such as EtOAc, MeCl₂, ACN, THF, and the like, to give (R)-toluene-4-sulfonic acid 2-methyl-butyl ester, (R)-toluene-4-sulfonic acid 2-methyl-pentyl ester, (R)-toluene-4-sulfonic acid 2-methyl-hexyl ester, and (R)-toluene-4-sulfonic acid 2,4-dimethyl-pentyl ester. Likewise, compounds of Formula 2 may be reacted with MsCl in the presence of an aprotic solvent, such as MTBE, toluene, or MeCl₂, and a weak base, such as Et₃N, to give (R)-methanesulfonic acid 2-methyl-butyl ester, (R)-methanesulfonic acid 2-methyl-pentyl ester, (R)-methanesulfonic acid 2-methyl-hexyl ester, and (R)-methanesulfonic acid 2,4-dimethyl-pentyl ester.

Upon activation of the hydroxy moiety, the resulting intermediate (Formula 4) is reacted with a 2-cyano succinic acid diester (Formula 5) in the presence of a base and one or more solvents to give a 2-alkyl-2-cyano succinic acid diester (Formula 6). Representative compounds of Formula 5 include 2-cyano-succinic acid diethyl ester. Likewise, representative compounds of Formula 6 include (2′R)-2-cyano-2-(2′-methyl-butyl)-succinic acid diethyl ester, (2′R)-2-cyano-2-(2′-methyl-pentyl)-succinic acid diethyl ester, (2′R)-2-cyano-2-(2′-methyl-hexyl)-succinic acid diethyl ester, and (2′R)-2-cyano-2-(2′,4′-dimethyl-pentyl)-succinic acid diethyl ester.

The alkylation may be carried out at temperatures that range from about RT to reflux, from about 70° C. to 110° C., or from about 90° C. to about 100° C., using stoichiometric or excess amounts (e.g., about 1 eq to about 1.5 eq) of the base and the diester (Formula 5). Representative bases include Group 1 metal carbonates (e.g., Cs₂CO₃ and K₂CO₃), phosphates (e.g., K₃PO₄), and alkoxides (e.g., 21% NaOEt in EtOH), as well as hindered, non-nucleophilic bases, such as Et₃N, t-BuOK, DBN, DBU, and the like. The reaction mixture may comprise a single organic phase or may comprise an aqueous phase, an organic phase, and a phase-transfer catalyst (e.g., a tetraalkylammonium salt such as Bu4N⁺Br⁻). Representative organic solvents include polar protic solvents, such as MeOH, EtOH, i-PrOH, and other alcohols; polar aprotic solvents, such as EtOAc, i-PrOAc, THF, MeCl₂, and ACN; and non-polar aromatic and aliphatic solvents, such as toluene, heptane, and the like.

Following alkylation, the ester moiety that is directly attached to the second asymmetric carbon atom (see Formula 6) is cleaved to give a 3-cyano carboxylic acid ester (Formula 7), such as (5R)-3-cyano-5-methyl-heptanoic acid ethyl ester, (5R)-3-cyano-5-methyl-octanoic acid ethyl ester, (5R)-3-cyano-5-methyl-nonanoic acid ethyl ester, and (5R)-3-cyano-5,7-dimethyl-octanoic acid ethyl ester. The ester may be removed by reacting the diester (Formula 6) with a chloride salt (e.g., LiCl, NaCl, etc.) in a polar aprotic solvent, such as aqueous DMSO, NMP, and the like, at a temperature of about 135° C. or greater (i.e., Krapcho conditions). Higher temperatures (e.g., 150° C., 160° C., or higher) or the use of a phase transfer catalyst (e.g., Bu4N⁺Br⁻) may be used to reduce the reaction times to 24 hours or less. Typically, the reaction employs excess chloride salt (e.g., from about 1.1 eq to about 4 eq or from about 1.5 eq to about 3.5 eq).

As shown in Scheme I and as noted above, the 3-cyano carboxylic acid ester (Formula 7) may be converted to the desired product (Formula 1) through contact with a resolving agent. In this method, the ester (Formula 7) is hydrolyzed via contact with an aqueous acid or base to give a 3-cyano carboxylic acid (Formula 8) or salt. For example, the compound of Formula 7 may be treated with HCl, H₂SO₄, and the like, and with excess H₂O to give the carboxylic acid of Formula 8. Alternatively, the compound of Formula 7 may be treated with an aqueous inorganic base, such as LiOH, KOH, NaOH, CsOH, Na₂CO₃, K₂CO₃, Cs₂CO₃, and the like, in an optional polar solvent (e.g., THF, MeOH, EtOH, acetone, ACN, etc.) to give a base addition salt, which may be treated with an acid to generate the 3-cyano carboxylic acid (Formula 8). Representative compounds of Formula 8 include (5R)-3-cyano-5-methyl-heptanoic acid, (5R)-3-cyano-5-methyl-octanoic acid, (5R)-3-cyano-5-methyl-nonanoic acid, and (5R)-3-cyano-5,7-dimethyl-octanoic acid, and their salts.

The cyano moiety of the carboxylic acid (Formula 8), or of its corresponding salt, is subsequently reduced to give, upon acid workup if necessary, a γ-amino acid (Formula 9). The penultimate free acid may be obtained by treating a salt of the γ-amino acid with a weak acid, such as aq HOAc. Representative compounds of Formula 9 include (5R)-3-aminomethyl-5-methyl-heptanoic acid, (5R)-3-aminomethyl-5-methyl-octanoic acid, (5R)-3-aminomethyl-5-methyl-nonanoic acid, and (5R)-3-aminomethyl-5,7-dimethyl-octanoic acid, and their salts.

The cyano moiety may be reduced via reaction with H₂ in the presence of a catalyst or through reaction with a reducing agent, such as LiAlH₄, BH₃-Me₂S, and the like. In addition to Raney nickel and other sponge metal catalysts, potentially useful catalysts include heterogeneous catalysts containing from about 0.1% to about 20%, or from about 1% to about 5%, by weight, of transition metals such as Ni, Pd, Pt, Rh, Re, Ru, and Ir, including oxides and combinations thereof, which are typically supported on various materials, including Al₂O₃, C, CaCO₃, SrCO3, BaSO₄, MgO, SiO₂, TiO₂, ZrO2, and the like. Many of these metals, including Pd, may be doped with an amine, sulfide, or a second metal, such as Pb, Cu, or Zn. Exemplary catalysts thus include palladium catalysts such as Pd/C, Pd/SrCO3, Pd/Al₂O₃, Pd/MgO, Pd/CaCO₃, Pd/BaSO₄, PdO, Pd black, PdCl₂, and the like, containing from about 1% to about 5% Pd, based on weight. Other catalysts include Rh/C, Ru/C, Re/C, PtO2, Rh/C, RUO₂, and the like.

The catalytic reduction of the cyano moiety is typically carried out in the presence of one or more polar solvents, including without limitation, water, alcohols, ethers, esters and acids, such as MeOH, EtOH, IPA, THF, EtOAc, and HOAc. The reaction may be carried out at temperatures ranging from about 5° C. to about 100° C., though reactions at RT are common. Generally, the substrate-to-catalyst ratio may range from about 1:1 to about 1000:1, based on weight, and H₂ pressure may range from about atmospheric pressure, 0 psig, to about 1500 psig. More typically, the substrate-to-catalyst ratios range from about 4:1 to about 20:1, and H₂ pressures range from about 25 psig to about 150 psig.

As shown in Scheme I, the penultimate γ-amino acid (Formula 9) is resolved to give the desired stereoisomer (Formula 1). The amino acid (Formula 9) may be resolved through contact with a resolving agent, such as an enantiomerically pure or substantially pure acid or base (e.g., S-mandelic acid, S-tartaric acid, and the like) to yield a pair of diastereoisomers (e.g., salts having different solubilities), which are separated via, e.g., recrystallization or chromatography. The γ-amino acid having the desired stereochemical configuration (Formula 1) is subsequently regenerated from the appropriate diastereomer via, e.g., contact with a base or acid or through solvent splitting (e.g., contact with EtOH, THF, and the like). The desired stereoisomer may be further enriched through multiple recrystallizations in a suitable solvent.

Besides using a resolving agent, the 3-cyano carboxylic acid ester (Formula 7) may be converted to the desired product (Formula 1) through contact with an enzyme. As shown in Scheme I and as discussed above, one diastereomer of the monoester (Formula 7) is diastereoselectively hydrolyzed through contact with an enzyme, which results in a mixture containing a 3-cyano carboxylic acid (or ester) having the requisite stereochemical configuration at C-3 (Formula 10) and a 3-cyano carboxylic ester (or acid) having the opposite (undesired) stereochemical configuration at C-3 (Formula 11). Representative compounds of Formula 10 include (3S,5R)-3-cyano-5-methyl-heptanoic acid, (3S,5R)-3-cyano-5-methyl-octanoic acid, (3S,5R)-3-cyano-5-methyl-nonanoic acid, and (3S,5R)-3-cyano-5,7-dimethyl-octanoic acid, and salts thereof, as well as C₁₋₆ alkyl esters of the aforementioned compounds, including (3S,5R)-3-cyano-5-methyl-heptanoic acid ethyl ester, (3S,5R)-3-cyano-5-methyl-octanoic acid ethyl ester, (3S,5R)-3-cyano-5-methyl-nonanoic acid ethyl ester, and (3S,5R)-3-cyano-5,7-dimethyl-octanoic acid ethyl ester. Exemplary compounds of Formula 11 include (3R,5R)-3-cyano-5-methyl-heptanoic acid, (3R,5R)-3-cyano-5-methyl-octanoic acid, (3R,5R)-3-cyano-5-methyl-nonanoic acid, and (3R,5R)-3-cyano-5,7-dimethyl-octanoic acid, and salts thereof, as well as C₁₋₆ alkyl esters of the aforementioned compounds, including (3R,5R)-3-cyano-5-methyl-heptanoic acid ethyl ester, (3R,5R)-3-cyano-5-methyl-octanoic acid ethyl ester, (3R,5R)-3-cyano-5-methyl-nonanoic acid ethyl ester, and (3R,5R)-3-cyano-5,7-dimethyl-octanoic acid ethyl ester.

The choice of enzyme (biocatalyst) used to resolve the desired diastereomer (Formula 10) depends on the structures of the substrate (Formula 7) and the bioconversion product (Formula 10 or Formula 11). The substrate (Formula 7) comprises two diastereoisomers (Formula 13 and Formula 14) having opposite stereochemical configuration at C-3,

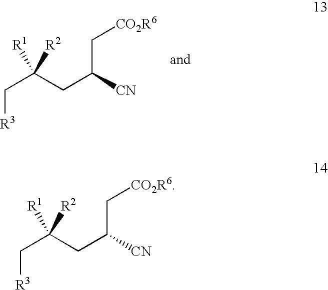

In Formula 13 and Formula 14, substituents R¹, R², and R⁶ are as defined for Formula 1 and Formula 5, above. The enzyme stereoselectively hydrolyzes one of the two diastereoisomers (Formula 13 or Formula 14). Thus, the enzyme may be any protein that, while having little or no effect on the compound of Formula 13, catalyzes the hydrolysis of the compound of Formula 14 to give a 3-cyano carboxylic acid (or salt) of Formula 11. Alternatively, the enzyme may be any protein that, while having little or no effect on the compound of Formula 14, catalyzes the hydrolysis of the compound of Formula 13 to give a 3-cyano carboxylic acid (or salt) of Formula 10. Useful enzymes for diastereoselectively hydrolyzing the compounds of Formula 13 or Formula 14 to compounds of Formula 10 or Formula 11, respectively, may thus include hydrolases, including lipases, certain proteases, and other stereoselective esterases. Such enzymes may be obtained from a variety of natural sources, including animal organs and microorganisms. See, e.g., Table 2 for a non-limiting list of commercially available hydrolases.

TABLE 2
Commercially Available Hydrolases

| Enzyme | Trade name |
|---|---|
| Porcine Pancreatic Lipase | Altus 03 |
| CAL-A, lyophilized | Altus 11 |
| Candida lipolytica Lipase | Altus 12 |
| CAL-B, lyophilized | Altus 13 |
| Geotrichum candidum Lipase | Altus 28 |
| Pseudomonas aroginosa Lipase | Altus 50 |
| Pseudomonas sp. Esterase | Amano Cholesterol Esterase 2 |
| Aspergillus niger Lipase | Amano Lipase AS |
| Burkholderia cepacia Lipase | Amano Lipase AH |
| Pseudomonas fluorescens Lipase | Amano Lipase AK 20 |
| Candida rugosa Lipase | Amano Lipase AYS |
| Rhizopus delemar Lipase | Amano Lipase D |
| Rhizopus oryzae Lipase | Amano Lipase F-AP 15 |
| Penicillium camembertii Lipase | Amano Lipase G 50 |
| Mucor javanicus Lipase | Amano Lipase M 10 |
| Burkholderia cepacia Lipase | Amano Lipase PS |
| Burkholderia cepacia Lipase | Amano Lipase PS-C I |
| Burkholderia cepacia Lipase | Amano Lipase PS-C II |
| Burkholderia cepacia Lipase | Amano Lipase PS-D I |
| Penicillium roqueforti Lipase | Amano Lipase R |
| Burkholderia cepacia Lipase | Amano Lipase S |
| Aspergillus sp. Protease | BioCatalytics 101 |
| Pseudomonas sp. Lipase | BioCatalytics 103 |
| Fungal Lipase | BioCatalytics 105 |
| Microbial, lyophilized Lipase | BioCatalytics 108 |
| CAL-B, lyophilized | BioCatalytics 110 |
| Candida sp., lyophilized | BioCatalytics 111 |
| CAL-A, lyophilized | BioCatalytics 112 |
| Thermomyces sp. Lipase | BioCatalytics 115 |
| Alcaligines sp., lyophilized Lipase | BioCatalytics 117 |
| Chromobacterium viscosum Lipase | Altus 26 |
| CAL-B, L2 Sol | Chriazyme L2 Sol |
| Candida cylindracea Lipase | Fluka 62302 |
| Candida utilis Lipase | Fluka 6 |
| Rhizopus niveus Lipase | Sigma L8 |
| Porcine Pancreatic Lipase | Sigma L12 |
| Pseudomonas sp. Lipoprotein Lipase | Sigma L13 |
| Thermomuces lanuginosus Lipase | Sigma L9 Lipolase |
| Thermomuces lanuginosus Lipase | Sigma L10 Novo871 |
| Rhizomucor miehei Lipase | Sigma L6 Palatase |
| Pseudomonas species Lipase | Sigma L14 Type XIII |
| Wheat Germ Lipase | Sigma L11 |
| Rhizopus arrhizus Lipase | Sigma L7 Type XI |
| Pancreatic Lipase 250 | Valley Research V1 |
| Trypsin Protease | Altus 33 |
| Chymopapain Protease | Altus 38 |
| Bromelain Protease | Altus 40 |
| Aspergillus niger Protease | Altus 41 |
| Aspergillus oryzae Protease | Altus 42 |
| Penicillium sp. Protease | Altus 43 |
| Aspergillus sp. Protease | Altus 45 |
| Renin Calf Stomach Protease | Sigma P24 |
| Subtilisin Carlsberg Protease | Altus 10 |
| Bacillus lentus Protease | Altus 53 |
| Fungal protease | Genencor Fungal Protease |
| | 500,000 |
| Fungal Protease | Genencor Fungal Protease |
| | Concentrate |
| Bacterial Protease | Genencor Protex 6L |
| Protease | Genencor Protease 899 |
| Bacterial protease | Genencor Multifect P3000 |
| Bacterial protease | Genencor Primatan |
| Bacterial protease | Genencor Purafect (4000L) |
| Bacterial protease | Genencor Multifect Neutral |
| Aspergillus niger Protease | Amano Acid Protease A |
| Rhizopus niveus Protease | Amano Acid Protease II |
| Rhizopus niveus Protease | Amano Newlase F |
| Rhizopus oryzae Protease | Amano Peptidase R |
| Bacillus subtilis Protease | Amano Proleather FGF |
| Aspergillus oryzae Protease | Amano Protease A |
| Aspergillus oryzae Protease | Amano Protease M |
| Bacillus subtilis Protease | Amano Protease N |
| Aspergillus melleus Protease | Amano Protease P 10 |
| Bacillus stearothermophilus Protease | Amano Protease SG |
| Pig Liver Esterase, lyophilized | BioCat Chirazyme E1 |
| Pig Liver Esterase, lyophilized | BioCat Chirazyme E2 |
| Streptomyces sp. Proteases | BioCatalytics 118 |
| Tritirachium album Protease | Fluka P6 Proteinase K |
| Bovine Pancreas Protease | Sigma P18 alpha chymotrypsin I |
| Streptomyces griseus Protease | Sigma P16 Bacterial |
| Bovine Pancreas Protease | Sigma P21 Beta chymotrypsin |
| Clostridium histolyticum Protease | Sigma P13 Clostripain |
| Bovine Intestine Protease | Sigma P17 Enteropeptidase |
| Porcine Intestine Protease | Sigma P25 Enteropeptidase |
| Bacillus sp. Protease | Sigma P8 Esperase |
| Aspergillus oryzae Protease | Sigma P1 Flavourzyme |
| Bacillus amyloliquefaciens Protease | Sigma P5 Neutrase |
| Carica papaya Protease | Sigma P12 Papain |
| Bacillus thermoproteolyticus rokko | Sigma P10 Protease |
| Pyrococcus furiosis Protease | Sigma P14 Protease S |
| Bacillus sp. Protease | Sigma P9 Savinase |
| Bovine Pancreas Protease | Sigma P19 Type 1 (crude) |
| Bacillus polymyxa Protease | Sigma P7 Type IX |
| Bacillus licheniformis Protease | Sigma P6 Type VIII |
| Aspergillus saitoi Protease | Sigma P3 Type XIII |
| Aspergillus sojae Protease | Sigma P4 Type XIX |
| Aspergillus oryzae Protease | Sigma P2 Type XXIII |
| Bacterial Protease | Sigma P11 Type XXIV |
| Rhizopus sp. Newlase | Sigma15 Newlase |
| Aspergillus oryzae Protease | Validase FP Concentrate |
| Pineapple [Ananas comosus & Ananas | Bromelian Concentrate |
| bracteatus (L)] | |
| Aspergillus sp. Acylase | Amano Am1 |
| Porcine kidney Acylase | Sigma A-S2 Acylase I |
| Penicillin G Acylase | Altus 06 |
| Esterase from Mucor meihei | Fluka E5 |
| Candida rugosa Esterase | Altus 31 |
| Porcine Pancreatic Elastase | Altus 35 |
| Cholinesterase, acetyl | Sigma ES8 |
| Cholesterol Esterase | BioCatalytics E3 |
| PLE - Ammonium Sulfate | BioCatalytics 123 |
| Rabbit Liver Esterase | Sigma ES2 |
| Cholesterol Esterase Pseudomonas sp. | Sigma ES4 |

As shown in the Example section, useful enzymes for the diastereoselective conversion of the cyano-substituted ester (Formula 13 or Formula 14) to the carboxylic acid (or salt) of Formula 10 or Formula 11 include lipases. Particularly useful lipases for conversion of the cyano-substituted ester of Formula 14 to a carboxylic acid (or salt) of Formula 11 include enzymes derived from the microorganism Burkholderia cepacia (formerly Pseudomonas cepacia), such as those available from Amano Enzyme Inc. under the trade names PS, PS-C I, PS-C II, PS-D I, and S. These enzymes are available as free-flowing powder (PS) or as lyophilized powder (S) or may be immobilized on ceramic particles (PS-C I and PS-C II) or diatomaceous earth (PS-D I). They have lypolytic activity that may range from about 30 KLu/g (PS) to about 2,200 KLu/g (S).

Particularly useful lipases for the conversion of the cyano-substituted ester of Formula 13 to a carboxylic acid (or salt) of Formula 10 include enzymes derived from the microorganism Thermomyces lanuginosus, such as those available from Novo-Nordisk A/S under the trade name LIPOLASE®. LIPOLASE® enzymes are obtained by submerged fermentation of an Aspergillus oryzae microorganism genetically modified with DNA from Thermomyces lanuginosus DSM 4109 that encodes the amino acid sequence of the lipase. LIPOLASE® 100L and LIPOLASE® 100T are available as a liquid solution and a granular solid, respectively, each having a nominal activity of 100 kLU/g. Other forms of LIPOLASE® include LIPOLASE® 50L, which has half the activity of LIPOLASE® 100L, and LIPOZYME® 100L, which has the same activity of LIPOLASE® 100L, but is food grade.

Various screening techniques may be used to identify suitable enzymes. For example, large numbers of commercially available enzymes may be screened using high throughput screening techniques described in the Example section below. Other enzymes (or microbial sources of enzymes) may be screened using enrichment isolation techniques. Such techniques typically involve the use of carbon-limited or nitrogen-limited media supplemented with an enrichment substrate, which may be the substrate (Formula 7) or a structurally similar compound. Potentially useful microorganisms are selected for further investigation based on their ability to grow in media containing the enrichment substrate. These microorganisms are subsequently evaluated for their ability to stereoselectively catalyze ester hydrolysis by contacting suspensions of the microbial cells with the unresolved substrate and testing for the presence of the desired diastereoisomer (Formula 10) using analytical methods such as chiral HPLC, gas-liquid chromatography, LC/MS, and the like.

Once a microorganism having the requisite hydrolytic activity has been isolated, enzyme engineering may be employed to improve the properties of the enzyme it produces. For example, and without limitation, enzyme engineering may be used to increase the yield and the diastereoselectivity of the ester hydrolysis, to broaden the temperature and pH operating ranges of the enzyme, and to improve the enzyme's tolerance to organic solvents. Useful enzyme engineering techniques include rational design methods, such as site-directed mutagenesis, and in vitro-directed evolution techniques that utilize successive rounds of random mutagenesis, gene expression, and high throughput screening to optimize desired properties. See, e.g., K. M. Koeller & C. -H. Wong, “Enzymes for chemical synthesis,” Nature 409:232-240 (11 Jan. 2001), and references cited therein, the complete disclosures of which are herein incorporated by reference.

The enzyme may be in the form of whole microbial cells, permeabilized microbial cells, extracts of microbial cells, partially purified enzymes, purified enzymes, and the like. The enzyme may comprise a dispersion of particles having an average particle size, based on volume, of less than about 0.1 mm (fine dispersion) or of about 0.1 mm or greater (coarse dispersion). Coarse enzyme dispersions offer potential processing advantages over fine dispersions. For example, coarse enzyme particles may be used repeatedly in batch processes, or in semi-continuous or continuous processes, and may usually be separated (e.g., by filtration) from other components of the bioconversion more easily than fine dispersions of enzymes.

Useful coarse enzyme dispersions include cross-linked enzyme crystals (CLECs) and cross-linked enzyme aggregates (CLEAs), which are comprised primarily of the enzyme. Other coarse dispersions may include enzymes immobilized on or within an insoluble support. Useful solid supports include polymer matrices comprised of calcium alginate, polyacrylamide, EUPERGIT®, and other polymeric materials, as well as inorganic matrices, such as CELITE®. For a general description of CLECs and other enzyme immobilization techniques, see U.S. Pat. No. 5,618,710 to M. A. Navia & N. L. St. Clair. For a general discussion of CLEAs, including their preparation and use, see U.S. Patent Application No. 2003/0149172 to L. Cao & J. Elzinga et al. See also A. M. Anderson, Biocat. Biotransform, 16:181 (1998) and P. López-Serrano et al., Biotechnol. Lett. 24:1379-83 (2002) for a discussion of the application of CLEC and CLEA technology to a lipase. The complete disclosures of the abovementioned references are herein incorporated by reference for all purposes.

The reaction mixture may comprise a single phase or may comprise multiple phases (e.g., a two- or a three-phase system). Thus, for example, the diastereoselective hydrolysis shown in Scheme I may take place in a single aqueous phase, which contains the enzyme, the substrate (Formula 7), the desired diastereomer (Formula 10), and the undesired diastereomer (Formula 11). Alternatively, the reaction mixture may comprise a multi-phase system that includes an aqueous phase in contact with a solid phase (e.g., enzyme or product), an aqueous phase in contact with an organic phase, or an aqueous phase in contact with an organic phase and a solid phase. For example, the diastereoselective hydrolysis may be carried out in a two-phase system comprised of a solid phase, which contains the enzyme, and an aqueous phase, which contains the substrate (Formula 7), the desired diastereomer (Formula 10), and the undesired diastereomer (Formula 11).

Alternatively, the diastereoselective hydrolysis may be carried out in a three-phase system comprised of a solid phase, which contains the enzyme, an organic phase that contains the substrate (Formula 7), and an aqueous phase that initially contains a small fraction of the substrate. In some cases the desired diastereomer (Formula 10) is a carboxylic acid which has a lower pKa than the unreacted ester (Formula 14). Because the carboxylic acid exhibits greater aqueous solubility, the organic phase becomes enriched in the unreacted ester (Formula 14) while the aqueous phase becomes enriched in the desired carboxylic acid (or salt). In other cases the undesired diastereomer (Formula 11) is a carboxylic acid, so the organic phase becomes enriched in the desired unreacted ester (Formula 13) while the aqueous phase becomes enriched in the undesired carboxylic acid (or salt).

The amounts of the substrate (Formula 7) and the biocatalyst used in the stereoselective hydrolysis will depend on, among other things, the properties of the particular cyano-substituted ester and the enzyme. Generally, however, the reaction may employ a substrate having an initial concentration of about 0.1 M to about 5.0 M, and in many cases, having an initial concentration of about 0.1 M to about 1.0 M. Additionally, the reaction may generally employ an enzyme loading of about 1% to about 20%, and in many cases, may employ an enzyme loading of about 5% to about 15% (w/w).

The stereoselective hydrolysis may be carried out over a range of temperature and pH. For example, the reaction may be carried out at temperatures of about 10° C. to about 60° C., but is typically carried out at temperatures of about RT to about 45° C. Such temperatures generally permit substantially full conversion (e.g., about 42% to about 50%) of the substrate (Formula 7) with a de (3S,5R diastereomer) of about 80% or greater (e.g., 98%) in a reasonable amount of time (e.g., about I h to about 48 h or about 1 h to about 24 h) without deactivating the enzyme. Additionally, the stereoselective hydrolysis may be carried out at a pH of about 5 to a pH of about 11, more typically at a pH of about 6 to a pH of about 9, and often at a pH of about 6.5 to a pH of about 7.5.

In the absence of pH control, the reaction mixture pH will decrease as the hydrolysis of the substrate (Formula 7) proceeds because of the formation of a carboxylic acid (Formula 10 or Formula 11). To compensate for this change, the hydrolysis reaction may be run with internal pH control (i.e., in the presence of a suitable buffer) or may be run with external pH control through the addition of a base. Suitable buffers include potassium phosphate, sodium phosphate, sodium acetate, ammonium acetate, calcium acetate, BES, BICINE, HEPES, MES, MOPS, PIPES, TAPS, TES, TRICINE, Tris, TRIZMA®, or other buffers having a pKa of about 6 to a pKa of about 9. The buffer concentration generally ranges from about 5 mM to about 1 mM, and typically ranges from about 50 mM to about 200 mM. Suitable bases include aqueous solutions comprised of KOH, NaOH, NH₄OH, etc., having concentrations ranging from about 0.5 M to about 15 M, or more typically, ranging from about 5 M to about 10 M. Other inorganic additives such as calcium acetate may also be used.

Following or during the enzymatic conversion of the substrate (Formula 7), the desired diastereomer (Formula 10) is isolated from the product mixture using standard techniques. For example, in the case of a single (aqueous) phase batch reaction, the product mixture may be extracted one or more times with an organic solvent, such as hexane, heptane, MeCl₂, toluene, MTBE, THF, etc., which separates the acid (ester) having the requisite stereochemical configuration at C-3 (Formula 10) from the undesirable ester (acid) (Formula 11) in the aqueous (organic) and organic (aqueous) phases, respectively. Alternatively, in the case of a multi-phase reaction employing aqueous and organic phases enriched in the acid or ester, the two diastereomers (Formula 10 and Formula 11) may be separated batch-wise following reaction, or may be separated semi-continuously or continuously during the stereoselective hydrolysis.

As shown in Scheme I, once the desired diastereomer (Formula 10) is isolated from the product mixture, it is optionally hydrolyzed using conditions and reagents associated with the ester hydrolysis of the compound of Formula 7, above. The cyano moiety of the resulting carboxylic acid (Formula 12), or its corresponding salt, is subsequently reduced to give, upon acid workup if necessary, the desired γ-amino acid (Formula 1). The reduction may employ the same conditions and reagents described above for reduction of the cyano moiety of the compound of Formula 8 and may be undertaken without isolating the cyano acid of Formula 12. Representative compounds of Formula 12 include (3S,5R)-3-cyano-5-methyl-heptanoic acid, (3S,5R)-3-cyano-5-methyl-octanoic acid, (3S,5R)-3-cyano-5-methyl-nonanoic acid, and (3S,5R)-3-cyano-5,7-dimethyl-octanoic acid, and their salts.

The chiral alcohol (Formula 2) shown in Scheme I may be prepared using various methods. For example, the chiral alcohol may be prepared by stereoselective enzyme-mediated hydrolysis of a racemic ester using conditions and reagents described above in connection with the enzymatic resolution of the compound of Formula 7. For example, n-decanoic acid 2-methyl-pentyl ester may be hydrolyzed in the presence of a hydrolase (e.g., lipase) and water to give a pure (or substantially pure) chiral alcohol, (R)-2-methyl-pentan-1-ol, which may be separated from the non-chiral acid and the unreacted chiral ester (n-decanoic acid and (S)-pentanoic acid 2-methyl-pentyl ester) by fractional distillation. The ester substrate may be prepared from the corresponding racemic alcohol (e.g., 2-methyl-pentan-1-ol) and acid chloride (e.g., n-decanoic acid chloride) or anhydride using methods known in the art.

Alternatively, the chiral alcohol (Formula 2) may be prepared by asymmetric synthesis of an appropriately substituted 2-alkenoic acid. For example, 2-methyl-pent-2-enoic acid (or its salt) may be hydrogenated in the presence of a chiral catalyst to give (R)-2-methyl-pentaonic acid or a salt thereof, which may be reduced directly with LAH to give (R)-2-methyl-pentan-1-ol or converted to the mixed anhydride or acid chloride and then reduced with NaBH₄ to give the chiral alcohol. Potentially useful chiral catalysts include cyclic or acyclic, chiral phosphine ligands (e.g., monophosphines, bisphosphines, bisphospholanes, etc.) or phosphinite ligands bound to transition metals, such as ruthenium, rhodium, iridium or palladium. Ru-, Rh-, Ir- or Pd-phosphine, phosphinite or phosphino oxazoline complexes are optically active because they possess a chiral phosphorus atom or a chiral group connected to a phosphorus atom, or because in the case of BINAP and similar atropisomeric ligands, they possess axial chirality.

Exemplary chiral ligands include BisP*; (R)-BINAPINE; (S)-Me-ferrocene-Ketalphos, (R,R)-DIOP; (R,R)-DIPAMP; (R)-(S)-BPPFA; (S,S)-BPPM; (+)-CAMP; (S,S)-CHIRAPHOS; (R)-PROPHOS; (R,R)-NORPHOS; (R)-BINAP; (R)-CYCPHOS; (R,R)-BDPP; (R,R)-DEGUPHOS; (R,R)-Me-DUPHOS; (R,R)-Et-DUPHOS; (R,R)-i-Pr-DUPHOS; (R,R)-Me-BPE; (R,R)-Et-BPE (R)-PNNP; (R)-BICHEP; (R,S,R,S)-Me-PENNPHOS; (S,S)-BICP; (R,R)-Et-FerroTANE; (R,R)-t-butyl-miniPHOS; (R)-Tol-BINAP; (R)-MOP; (R)-QUINAP; CARBOPHOS; (R)-(S)-JOSIPHOS; (R)-PHANEPHOS; BIPHEP; (R)-Cl-MeO-BIPHEP; (R)-MeO-BIPHEP; (R)-MonoPhos; BIFUP; (R)-SpirOP; (+)-TMBTP; (+)-tetraMeBITIANP; (R,R,S,S) TANGPhos; (R)-PPh₂-PhOx-Ph; (S,S) MandyPhos; (R)-eTCFP; (R)-mTCFP; and (R)-CnTunaPHOS, where n is an integer of 1 to 6.

Other chiral ligands include (R)-(−)-1-[(S)-2-(di(3,5-bis-trifluoromethylphenyl)phosphino)ferrocen-yl]ethyldicyclohexyl-phosphine; (R)-(−)-1-[(S)-2-(di(3,5-bis-trifluoromethylphenyl)phosphino)ferrocen-yl]ethyldi(3,5-dimethylphenyl)phosphine; (R)-(−)-1-[(S)-2-(di-t-butylphosphino)ferro-cenyl]ethyldi(3,5-dimethylphenyl)phosphine; (R)-(−)-1-[(S)-2-(dicyclohexylphbsphi-no)ferrocenyl]ethyldi-t-butylphosphine; (R)-(−)-1-[(S)-2-(dicyclohexylphosphino)ferrocenyl]ethyldicyclohexylphosphine; (R)-(−)-1-[(S)-2-(dicyclohexylphosphino)ferro-cenyl]ethyldiphenylphosphine; (R)-(−)-1-[(S)-2-(di(3,5-dimethyl-4-methoxyphen-yl)phosphino)ferrocenyl]ethyldicyclohexylphosphine; (R)-(−)-1-[(S)-2-(diphenylphos-phino)ferrocenyl]ethyldi-t-butylphosphine; (R)-N-[2-(N,N-dimethylamino)ethyl]-N-methyl-1-[(S)-1′,2-bis(diphenylphosphino)ferrocenyl]ethylamine; (R)-(+)-2-[2-(diphenylphosphino)phenyl]-4-(1-methylethyl)-4,5-dihydrooxazole; {1-[((R,R)-2-benzyl-phospholanyl)-phen-2-yl]-(R*,R*)-phospholan-2-yl}-phenyl-methane; and {1-[((R,R)-2-benzyl-phospholanyl)-ethyl]-(R*,R*)-phospholan-2-yl}-phenyl-methane.

Useful ligands may also include stereoisomers (enantiomers and diastereoisomers) of the chiral ligands described in the preceding paragraphs, which may be obtained by inverting all or some of the stereogenic centers of a given ligand or by inverting the stereogenic axis of an atropoisomeric ligand. Thus, for example, useful chiral ligands may also include (S)-Cl-MeO— BIPHEP; (S)-PHANEPHOS; (S,S)-Me-DUPHOS; (S,S)-Et-DUPHOS; (S)-BINAP; (S)-Tol-BINAP; (R)-(R)-JOSIPHOS; (S)-(S)-JOSIPHOS; (S)-eTCFP; (S)-mTCFP and so on.

Many of the chiral catalysts, catalyst precursors, or chiral ligands may be obtained from commercial sources or may be prepared using known methods. A catalyst precursor or pre-catalyst is a compound or set of compounds, which are converted into the chiral catalyst prior to use. Catalyst precursors typically comprise Ru, Rh, Ir or Pd complexed with the phosphine ligand and either a diene (e.g., norboradiene, COD, (2-methylallyl)₂, etc.) or a halide (Cl or Br) or a diene and a halide, in the presence of a counterion, X⁻, such as OTf⁻, PF₆⁻, BF₄⁻, SbF₆⁻, ClO₄⁻, etc. Thus, for example, a catalyst precursor comprised of the complex, [(bisphosphine ligand)Rh(COD)]⁺X⁻ may be converted to a chiral catalyst by hydrogenating the diene (COD) in MeOH to yield [(bisphosphine ligand)Rh(MeOH)2]⁺X⁻. MeOH is subsequently displaced by the enamide (Formula 2) or enamine (Formula 4), which undergoes enantioselective hydrogenation to the desired chiral compound (Formula 3). Examples of chiral catalysts or catalyst precursors include (+)-TMBTP-ruthenium(II) chloride acetone complex; (S)-Cl-MeO— BIPHEP-ruthenium(II) chloride Et₃N complex; (S)-BINAP-ruthenium(II) Br₂ complex; (S)-tol-BINAP-ruthenium(II) Br₂ complex; [((3R,4R)-3,4-bis(diphenylphosphino)-1-methylpyrrolidine)-rhodium-(1,5-cyclooctadiene)]-tetrafluoroborate complex; [((R,R,S,S)-TANGPhos)-rhodium(I)-bis(1,5-cyclooctadiene)]-trifluoromethane sulfonate complex; [(R)-B INAPINE-rhodium-(1,5-cyclooctaidene)]-tetrafluoroborate complex; [(S)-eTCFP-(1,5-cyclooctadiene)-rhodium(I)]-tetrafluoroborate complex; and [(S)-mTCFP-(1,5-cyclooctadiene)-rhodium(I)]-tetrafluroborate complex.

For a given chiral catalyst and hydrogenation substrate, the molar ratio of the substrate and catalyst (s/c) may depend on, among other things, H₂ pressure, reaction temperature, and solvent (if any). Usually, the substrate-to-catalyst ratio exceeds about 100:1 or 200:1, and substrate-to-catalyst ratios of about 1000:1 or 2000:1 are common. Although the chiral catalyst may be recycled, higher substrate-to-catalyst ratios are more useful. For example, substrate-to-catalyst ratios of about 1000:1, 10,000:1, and 20,000:1, or greater, would be useful. The asymmetric hydrogenation is typically carried out at about RT or above, and under about 10 kPa (0.1 atm) or more of H₂. The temperature of the reaction mixture may range from about 20° C. to about 80° C., and the H₂ pressure may range from about 10 kPa to about 5000 kPa or higher, but more typically, ranges from about 10 kPa to about 100 kPa. The combination of temperature, H₂ pressure, and substrate-to-catalyst ratio is generally selected to provide substantially complete conversion (i.e., about 95 wt %) of the substrate (Formula 2 or 4) within about 24 h. With many of the chiral catalysts, decreasing the H₂ pressure increases the enantioselectivity.

A variety of solvents may be used in the asymmetric hydrogenation, including protic solvents, such as water, MeOH, EtOH, and i-PrOH. Other useful solvents include aprotic polar solvents, such as THF, ethyl acetate, and acetone. The stereoselective hydrogenation may employ a single solvent or may employ a mixture of solvents, such as THF and MeOH, THF and water, EtOH and water, MeOH and water, and the like.

The compound of Formula 1, or its diastereoisomers, may be further enriched through, e.g., fractional recrystallization or chromatography or by recrystallization in a suitable solvent.

As described throughout the specification, many of the disclosed compounds have stereoisomers. Some of these compounds may exist as single enantiomers (enantiopure compounds) or mixtures of enantiomers (enriched and racemic samples), which depending on the relative excess of one enantiomer over another in a sample, may exhibit optical activity. Such stereoisomers, which are non-superimposable mirror images, possess a stereogenic axis or one or more stereogenic centers (i.e., chirality). Other disclosed compounds may be stereoisomers that are not mirror images. Such stereoisomers, which are known as diastereoisomers, may be chiral or achiral (contain no stereogenic centers). They include molecules containing an alkenyl or cyclic group, so that cis/trans (or Z/E) stereoisomers are possible, or molecules containing two or more stereogenic centers, in which inversion of a single stereogenic center generates a corresponding diastereoisomer. Unless stated or otherwise clear (e.g., through use of stereobonds, stereocenter descriptors, etc.) the scope of the present invention generally includes the reference compound and its stereoisomers, whether they are each pure (e.g., enantiopure) or mixtures (e.g., enantiomerically enriched or racemic).

Some of the compounds may also contain a keto or oxime group, so that tautomerism may occur. In such cases, the present invention generally includes tautomeric forms, whether they are each pure or mixtures.

Many of the compounds described herein are capable of forming pharmaceutically acceptable salts. These salts include acid addition salts (including di-acids) and base salts. Pharmaceutically acceptable acid addition salts include nontoxic salts derived from inorganic acids such as hydrochloric, nitric, phosphoric, sulfuric, hydrobromic, hydroiodic, hydrofluoric, phosphorous, and the like, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, malate, tartrate, methanesulfonate, and the like.

Pharmaceutically acceptable base salts include nontoxic salts derived from bases, including metal cations, such as an alkali or alkaline earth metal cation, as well as amines. Examples of suitable metal cations include sodium cations (Na⁺), potassium cations (K⁺), magnesium cations (Mg²⁺), calcium cations (Ca²⁺), and the like. Examples of suitable amines include N,N′-dibenzylethylenediamine, chloroprocaine, choline, diethanolamine, dicyclohexylamine, ethylenediamine, N-methylglucamine, and procaine. For a discussion of useful acid addition and base salts, see S. M. Berge et al., “Pharmaceutical Salts,” 66 J. of Pharm. Sci., 1-19 (1977); see also Stahl and Wermuth, Handbook of Pharmaceutical Salts: Properties, Selection, and Use (2002).

One may prepare an acid addition salt (or base salt) by contacting a compound's free base (or free acid) with a sufficient amount of a desired acid (or base) to produce a nontoxic salt. One may then isolate the salt by filtration if it precipitates from solution, or by evaporation to recover the salt. One may also regenerate the free base (or free acid) by contacting the acid addition salt with a base (or the base salt with an acid). Certain physical properties (e.g., solubility, crystal structure, hygroscopicity, etc.) of a compound's free base, free acid, or zwitterion may differ from its acid or base addition salt. Generally, however, references to the free acid, free base or zwitterion of a compound would include its acid and base addition salts.

Disclosed and claimed compounds may exist in both unsolvated and solvated forms and as other types of complexes besides salts. Useful complexes include clathrates or compound-host inclusion complexes where the compound and host are present in stoichiometric or non-stoichiometric amounts. Useful complexes may also contain two or more organic, inorganic, or organic and inorganic components in stoichiometric or non-stoichiometric amounts. The resulting complexes may be ionized, partially ionized, or non-ionized. For a review of such complexes, see J. K. Haleblian, J. Pharm. Sci. 64(8): 1269-88 (1975). Pharmaceutically acceptable solvates also include hydrates and solvates in which the crystallization solvent may be isotopically substituted, e.g. D₂O, d₆-acetone, d₆-DMSO, etc. Generally, for the purposes of this disclosure, references to an unsolvated form of a compound also include the corresponding solvated or hydrated form of the compound.

The disclosed compounds also include all pharmaceutically acceptable isotopic variations, in which at least one atom is replaced by an atom having the same atomic number, but an atomic mass different from the atomic mass usually found in nature. Examples of isotopes suitable for inclusion in the disclosed compounds include isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of fluorine, such as ¹⁸F; and isotopes of chlorine, such as ³⁶Cl. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies.

### EXAMPLES

The following examples are intended to be illustrative and non-limiting, and represent specific embodiments of the present invention.

General Materials and Methods

Enzyme screening was carried out using a 96-well plate, which is described in D. Yazbeck et al., Synth. Catal. 345:524-32 (2003), the complete disclosure of which is herein incorporated by reference for all purposes. All enzymes used in the screening plate (see Table 2) were obtained from commercial enzyme suppliers including Amano Enzyme Inc. (Nagoya, Japan), Roche (Basel, Switzerland), Novo Nordisk (Bagsvaerd, Denmark), Altus Biologics Inc. (Cambridge, Mass.), Biocatalytics (Pasadena, Calif.), Toyobo (Osaka, Japan), Sigma-Aldrich (St. Louis, Mo.), Fluka (Buchs, Switzerland), Genencor International, Inc. (Rochester, N.Y.), and Valley Research (South Bend, Ind.). The screening reactions were performed in an Eppendorf Thermomixer-R (VWR). Subsequent larger scale enzymatic resolutions employed LIPOLASE® 100L EX, which is available form Novo-Nordisk A/S (CAS no. 9001-62-1), as well as Lipase PS, PS-C I, PS-C II, and PS-D I, which are available from Amano Enzyme Inc.

### Example 1

### Preparation of (R)-methanesulfonic acid 2-methyl-pentyl ester

A 4000 L reactor was charged with (R)-2-methyl-pentan-1-ol (260 kg, 2500 mol), MTBE (2000 L), and cooled to −10° C. to 0° C. Methanesulfonyl chloride (310 kg, 2600 mol) was charged, and then Et₃N (310 kg, 3100 mol) was added while maintaining the internal temperature at 0° C. to 10° C. After the addition was complete, the reaction mixture was warmed to 15° C. to 25° C. and stirred at this temperature for at least 1 h until complete by GC analysis. A solution of aq HCl (88 kg of HCl in 700 L of water) was then added to the reaction mixture. The resulting mixture stirred for at least 15 min, settled for at least 15 min, and then the lower aqueous phase was removed. The upper organic phase was washed with water (790 L) and aqueous sodium bicarbonate (67 kg of sodium bicarbonate in 840 L of water). The solution was then concentrated under vacuum to remove the MTBE to afford the titled compound as an oil (472 kg, 95% yield). ¹H NMR (400 MHz, CDCl₃) 4.07-3.93 ppm (m, 2H), 2.97 (s, 3H), 1.91-1.80 (m, 1H), 1.42-1.09 (m, 4H), 0.94 (d, J=6.57 Hz, 3H), 0.87 (t, J=6.56 Hz, 3H); ¹³C NMR (CDCl₃) 74.73, 37.01, 34.81, 32.65, 19.71, 16.29, 14.04.

### Example 2

### Preparation of (2′R)-2-cyano-2-(2′-methyl-pentyl)-succinic acid diethyl ester

A 4000 L reactor was charged with (R)-methanesulfonic acid 2-methyl-pentyl ester (245 kg, 1359 mol), 2-cyano-succinic acid diethyl ester (298 kg, 1495 mol), and anhydrous EtOH (1300 kg). Sodium ethoxide (506 kg, 21 wt % in EtOH) was added. The resulting solution was heated to 70° C. to 75° C., and the mixture stirred at this temperature for at least 18 h until complete by GC analysis. After the reaction was complete, a solution of aqueous HCl (32 kg of HCl in 280 L of water) was then added to the reaction mixture until the pH was <2. Additional water (400 L) was added, and the reaction mixture was then concentrated under vacuum to remove the ethanol. MTBE (1000 kg) was added, and the mixture was stirred for at least 15 min, settled for at least 15 min, and then the lower aqueous layer was back extracted with MTBE (900 kg). The combined organic phases were concentrated under vacuum to afford the titled compound as a dark oil (294 kg, 79% yield corrected for purity). ¹H NMR (400 MHz, CDC₁₃) 4.29 ppm (q, J=7.07 Hz, 2H), 4.18 (q, J=7.07 Hz, 2H), 3.03 (dd, J=6.6, 7.1 Hz, 2H), 1.93-1.61 (m, 3H), 1.40-1.20 (m, 10H), 0.95-0.82 (m, 6H); ¹³C NMR (CDCl₃) 168.91, 168.67, 168.59, 168.57, 119.08, 118.82, 62.95, 62.90, 44.32, 44.19, 42.21, 42.02, 39.77, 39.64, 30.05, 29.91, 20.37, 19.91, 19.66, 13.99.

### Example 3

### Preparation of (SR)-3-cyano-5-methyl-octanoic acid ethyl ester (Method A)

A 4000 L reactor was charged with NaCi (175 kg, 3003 mol), tetrabutylammonium bromide (33.1 kg, 103 mol), water (87 L), and DMSO (1000 kg). (2′R)-2-Cyano-2-(2′-methyl-pentyl)-succinic acid diethyl ester (243 kg, 858 mol) was charged and the mixture was heated to 135° C. to 138° C. and stirred at this temperature for at least 48 h, until complete by GC analysis. After the reaction was cooled to 25° C. to 35° C., heptane (590 kg) was added, and the mixture stirred for at least 15 min, settled for at least 15 min, and then the lower aqueous phase was removed. The upper organic phase was washed with water (800 L). The heptane solution containing the product was decolorized with carbon, and concentrated under vacuum to afford the titled compound as an orange oil (133.9 kg, 74% yield corrected for purity). ¹H NMR (400 MHz, CDCl₃) 4.20 ppm (q, J=7.07 Hz, 2H), 3.13-3.01 (m, 1H), 2.75-2.49 (m, 2H), 1.80-1.06 (m, 10H), 0.98-086 (m, 6H); ¹³ C NMR (CDCl₃) 169.69, 169.65, 121.28, 120.99, 61.14, 39.38, 39.15, 38.98, 37.67, 37.23, 36.95, 30.54, 30.47, 25.67, 25.45, 19.78, 19.61, 19.53, 18.56, 14.13, 14.05.

### Example 4

### Preparation of (5R)-3-cyano-5-methyl-octanoic acid ethyl ester (Method B)

A 250 mL flask was charged with LiCl (3.89 g, 0.0918 mol), water (7 mL), and DMSO (72 mL). (2′R)-2-Cyano-2-(2′-methyl-pentyl)-succinic acid diethyl ester (25.4 g, 0.0706 mol, 78.74% by GC) was charged and the mixture was heated to 135° C. to 138° C. and stirred at this temperature for at least 24 h, until complete by GC analysis. After the reaction was cooled to 25° C. to 35° C., heptane (72 niL), saturated NaCl (72 mL), and water (72 mL) was added and the mixture stirred for at least 15 min, settled for at least 15 min, and then the lower aqueous phase was washed with heptane (100 mL). The combined organic phases were concentrated under vacuum to afford the titled compound as an orange oil (13.0 g, 84% yield corrected for purity).

### Example 5

### Preparation of (5R)-3-cyano-5-methyl-octanoic acid sodium salt

A 4000 L reactor was charged with (5R)-3-cyano-5-methyl-octanoic acid ethyl ester (250 kg, 1183 mol) and THF (450 kg). An aqueous solution of NaOH was prepared (190 kg of 50% NaOH in 350 L of water) and then added to the THF solution. The resulting solution was stirred at 20° C. to 30° C. for at least 2 h, until the reaction was complete by GC analysis. After this time, THF was removed by vacuum distillation to afford an aqueous solution of the titled compound, which was used immediately in the next step.

### Example 6

### Preparation of (5R)-3-aminomethyl-5-methyl-octanoic acid sodium salt

A 120 L autoclave was charged with sponge nickel catalyst (3.2 kg, Johnson & Mathey A7000) followed by an aqueous solution of (5R)-3-cyano-5-methyl-octanoic acid sodium salt (15 kg in 60 L of water) and the resulting mixture was hydrogenated under 50 psig of hydrogen at 30° C. to 35° C. for at least 18 h, or until hydrogen uptake ceased. The reaction was then cooled to 20° C. to 30° C., and the spent catalyst was removed by filtration through a 0.2μ filter. The filter cake was washed with water (2×22 L), and the resulting aqueous solution of the titled compound was used directly in the next step.

### Example 7

### Preparation of (5R)-3-aminomethyl-5-methyl-octanoic acid

A 4000 L reactor was charged with an aqueous solution of (5R)-3-aminomethyl-5-methyl-octanoic acid (˜150 kg in ˜1000 L of water) and cooled to 0° C. to 5° C. Glacial acetic acid was added until the pH was 6.3 to 6.8. To the mixture was added anhydrous EtOH (40 kg). The resulting slurry was heated to 65° C. to 70° C. for less than 20 min and was cooled to 0° C. to 5° C. over 3 h. The product was collected by filtration to afford the titled compound as a water-wet cake (76 kg, 97% yield corrected for purity, 10% water by KF), which was used in the next step. ¹H NMR (400 MHz, D₃COD) 4.97 ppm (BS, 3H), 3.00-2.74 (m, 2H), 2.48-2.02 (m, 3H), 1.61-1.03 (m, 7H), 0.94-086 (m, 6H); ¹³C NMR (D₃COD) 181.10, 181.07, 46.65, 45.86, 44.25, 43.15, 42.16, 41.64, 41.35, 33.45, 31.25, 31.20, 21.45, 21.41, 20.52, 20.12, 15.15, 15.12.

### Example 8

### Preparation of (3S,5R)-3-aminomethyl-5-methyl-octanoic acid via Contact with a Resolving Agent

A 4000 L reactor was charged with water wet (10%) (SR)-3-aminomethyl-5-methyl-octanoic acid (76 kg, 365 mol), (S)-mandelic acid (34.8 kg, 229 mol), anhydrous EtOH (1780 kg), and water (115 L). The resulting mixture was heated to 65° C. to 70° C. and stirred until the solids dissolved. The solution was then cooled to 0° C. to 5° C. over 2 h and stirred at this temperature for an additional 1 h. The product was collected by filtration, and the cake was washed with −20° C. EtOH (3×60 kg). The crude product (18 kg in 48% yield) and EtOH (167 kg) were charged to a reactor. The mixture was cooled to 0° C. to 5° C. and stirred at this temperature for 1.5 h. The product was then collected by filtration, and the cake was washed with −20° C. EtOH (3×183 kg) to afford the titled compound (17 kg, 94% yield). The quasimolecular ion (MH+) of the titled compound was observed at 188.1653 amu and is in agreement with the theoretical value of 188.1650; the measured value establishes the molecular formula as C₁₀H₂₁NO₂ as no reasonable alternate chemical entity containing only C, H, N, and O can exist with a molecular ion within the 5-ppm (0.9 mDa) experimental error of the measured value; IR (KBr) 2955.8 cm⁻¹, 22.12.1, 1643.8,1551.7, 1389.9; ¹H NMR (400 MHz, D₃COD) 4.91 ppm (bs, 2H), 3.01-2.73 (m, 2H), 2.45-2.22 (m, 2H), 1.60-1.48 (m, 1H), 1.45-1.04 (m, 6H), 0.98-086 (m, 6H); ¹³C NMR (D₃COD) 181.04, 45.91, 44.30, 42.13, 40.65, 33.42, 31.24, 21.39, 20.49, 15.11.

### Example 9

### Enzyme Screening via Enzymatic Hydrolysis of (5R)-3-cyano-5-methyl-octanoic acid ethyl ester (Formula 15) to Yield (3S,5R)-3-cyano-5-methyl-octanoic acid sodium salt (Formula 16, R¹⁰=Na⁺) and (3R,5R)-3-cyano-5-methyl-octanoic acid ethyl ester (Formula 17, R¹¹=Et) or (3S,5R)-3-cyano-5-methyl-octanoic acid ethyl ester (Formula 16, R¹⁰=Et) and (3R,5R)-3-cyano-5-methyl-octanoic acid sodium salt (Formula 17, R¹¹=Na⁺)

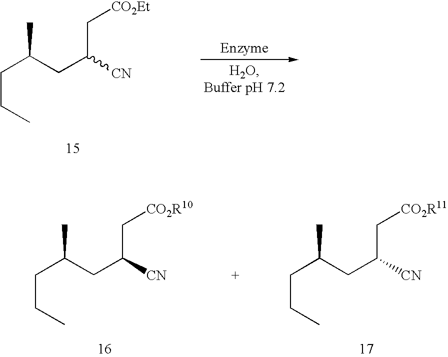

Enzyme screening was carried out using a screening kit comprised of individual enzymes deposited in separate wells of a 96-well plate, which was prepared in advance in accordance with a method described in D. Yazbeck et al., Synth. Catal. 345:524-32 (2003). Each of the wells has an empty volume of 0.3 mL (shallow well plate). One well of the 96-well plate contains only phosphate buffer (10 μL, 0.1 M, pH 7.2). With few exceptions, each of the remaining wells contain one aliquot of enzyme (10 μL, 83 mg/mL), most of which are listed in Table 2, above. Prior to use, the screening kit is removed from storage at −80° C. and the enzymes are allowed to thaw at RT for about 5 min. Potassium phosphate buffer (85 μL, 0.1 M, pH 7.2) is dispensed into each of the wells using a multi-channel pipette. Concentrated substrate (Formula 15, 5 μL) is subsequently added to each well via a multi-channel pipette and the 96 reaction mixtures are incubated at 30° C. and 750 rpm. The reactions are quenched and sampled after 24 h by transferring each of the reaction mixtures into separate wells of a second 96-well plate. Each of the wells has an empty volume of 2 mL (deep well plate) and contains EtOAc (1 mL) and HCl (1N, 100 μL). The components of each well are mixed by aspirating the well contents with a pipette. The second plate is centrifuged and 100 μL of the organic supernatant is transferred from each well into separate wells of a third 96-well plate (shallow plate). The wells of the third plate are subsequently sealed using a penetrable mat cover. Once the wells are sealed, the third plate is transferred to a GC system for determination of diastereoselectivity (de).

Table 3 lists enzyme, trade name, E value, χ, and selectivity for some of the enzymes that were screened. For a given enzyme, the E value may be interpreted as the relative reactivity of a pair of diastereomers (substrates). The E values listed in Table 3 were calculated from GC/derivatization data (fractional conversion, χ, and de) using a computer program called Ee2, which is available from the University of Graz. In Table 3, selectivity corresponds to the diastereomer —(3R,5R)-3-cyano-5-methyl-octanoic acid ethyl ester or (3S,5R)-3-cyano-5-methyl-octanoic acid ethyl ester— that underwent the greatest hydrolysis for a given enzyme.

TABLE 3
Results from Screening Reactions of Example 1

| Enzyme | Trade Name | E | χ | Selectivity |
|---|---|---|---|---|
| Porcine Pancreatic Lipase | Altus 03 | 1.5 | 15 | (3R,5R) |
| Candida cylindracea Lipase | Fluka 62302 | 1.4 | 3 | (3R,5R) |
| Burkholderia cepacia Lipase | Amano Lipase AH | 200 | 15 | (3R,5R) |
| Pseudomonas fluorescens Lipase | Amano Lipase AK 20 | 200 | 25 | (3R,5R) |
| Candida rugosa Lipase | Amano Lipase AYS | 1.4 | 2 | (3R,5R) |
| Rhizopus delemar Lipase | Amano Lipase D | 6 | 44 | (3S,5R) |
| Rhizopus oryzae Lipase | Amano Lipase F-AP 15 | 20 | 1 | (3S,5R) |
| Penicillium camembertii Lipase | Amano Lipase G 50 | 1.1 | 6 | (3S,5R) |
| Mucor javanicus Lipase | Amano Lipase M 10 | 8 | 3 | (3S,5R) |
| Burkholderia cepacia Lipase | Amano Lipase PS | 200 | 45 | (3R,5R) |
| Pseudomonas sp. Lipase | BioCatalytics 103 | 4 | 7 | (3S,5R) |
| Microbial, lyophilized Lipase | BioCatalytics 108 | 17 | 45 | (3R,5R) |
| CAL-B, lyophilized | BioCatalytics 110 | 1.2 | 96 | (3S,5R) |
| Candida sp., lyophilized | BioCatalytics 111 | 1.2 | 8 | (3R,5R) |
| CAL-A, lyophilized | BioCatalytics 112 | 1.6 | 5 | (3R,5R) |
| Thermomyces sp. Lipase | BioCatalytics 115 | 7 | 50 | (3S,5R) |
| Alcaligines sp., lyophilized Lipase | BioCatalytics 117 | 15 | 31 | (3R,5R) |
| CAL-B, L2 Sol | Chriazyme L2 Sol | 1.3 | 31 | (3R,5R) |
| Thermomuces lanuginosus Lipase | Sigma L9 Lipolase | 15 | 50 | (3S,5R) |
| Thermomuces lanuginosus Lipase | Sigma L10 Novo871 | 10 | 68 | (3S,5R) |
| Rhizomucor miehei Lipase | Sigma L6 Palatase | 5.3 | 90 | (3S,5R) |
| Fungal protease concentrate | Genencor | 10 | 10 | (3R,5R) |
| Bovine Pancreas Protease | Sigma P18 α-chymotrypsin I | 10 | 10 | (3R,5R) |
| Pineapple [Ananas comosus & | Bromelian Concentrate | 10 | 10 | (3R,5R) |
| Ananas bracteatus (L)] | | | | |
| Porcine kidney Acylase | Sigma A-S2 Acylase I | 2 | 60 | (3S,5R) |
| Esterase from Mucor meihei | Fluka E5 | 5 | 79 | (3S,5R) |
| Cholinesterase, acetyl | Sigma ES8 | 1.1 | 54 | (3S,5R) |
| Cholesterol Esterase | BioCatalytics E3 | 1.1 | 54 | (3S,5R) |
| PLE - Ammonium Sulfate | BioCatalytics 123 | 1.3 | 71 | (3S,5R) |

### Example 10

### Preparation of (3S,5R)-3-cyano-5-methyl-octanoic acid tert-butyl-ammonium salt via Enzymatic Resolution

To a 50 mL reactor equipped with a pH electrode, an overhead stirrer and a base addition line, was added (5R)-3-cyano-5-methyl-octanoic acid ethyl ester (8 g, 37.85 mmol), followed by calcium acetate solution (8 mL), deionized water (3.8 mL), and LIPOLASE® 100L EX (0.2 mL). The resulting suspension was stirred at room temperature for 24 h. The pH of the solution was maintained at 7.0 by adding 4M NaOH. The course of the reaction was tracked by GC (conversion and % de of the product and starting material), and was stopped after 45% of the starting material had been consumed (˜4.3 mL of NaOH had added). After reaction completion, toluene (20 mL) was added, and the mixture stirred for 1 min. The pH was lowered to 3.0 by adding concentrated HCl aq and the solution was stirred for 5 min and then transferred to a separatory funnel/extractor. The organic layer was separated and the aqueous layer extracted once with 10 mL of toluene. The organic layers were pooled and toluene evaporated to dryness. The crude product (sodium salt of (3S,5R)-3-cyano-5-methyl-octanoic acid, 75% de by GC) was re-suspended in MTBE (40 mL). Tert-butylamine (1.52 g, 1.1 eq) was added dropwise to the mixture with stirring over a 5 minute period. Crystals precipitated shortly after the addition was finished and they were collected in a buchner funnel. The solid was washed with MTBE (2×20 mL). The residue was then dried under vacuum to afford the titled compound (2.58 g, 96% de by GC).

### Example 11

### Resolution of (3S,5R)-3-cyano-5-methyl-octanoic acid ethyl ester via Enzymatic Hydrolysis of (3R,5R)-3-cyano-5-methyl-octanoic acid ethyl ester to (3R,5R)-3-cyano-5-methyl-octanoic acid sodium salt

To a vessel containing sodium phosphate (monobasic) monohydrate (4.7 kg) and water (1650 L) at a temperature of 20° C. to 25° C. is added 50% NaOH aq (2.0 kg). After stirring for 15 min, the pH of the mixture is checked to ensure that it is in the range of 6.0 to 8.0. Amano PS lipase (17 kg) is added and the mixture is stirred for 30 min to 60 min at 20° C. to 25° C. The mixture is filtered to remove solids and the filtrate is combined with sodium bicarbonate (51 kg), (5R)-3-cyano-5-methyl-octanoic acid ethyl ester (154 kg), and water (10 L). The mixture is allowed to react at about 50° C. for 24 h to 48 h. The course of the enzymatic hydrolysis is monitored by GC and is considered to be complete when the ratio of (3S,5R)-3-cyano-5-methyl-octanoic acid ethyl ester to (3R,5R)-3-cyano-5-methyl-octanoic acid sodium salt is greater than 99:1 based on GC. Following completion of the reaction, the mixture is added to a vessel charged with NaCl (510 kg), and the contents of the vessel are stirred at 20° C. to 25° C. The mixture is extracted with MTBE (680 L) and the aqueous and organic phases are separated. The aqueous phase is discarded and the organic phase is washed with NaCl (26 kg), sodium bicarbonate (2 kg), and water (85 L). After the solids are dissolved, the mixture is again extracted with MTBE (680 L), the aqueous and organic phases separated, and the organic phase is again washed with NaCl (26 kg), sodium bicarbonate (2 kg), and water (85 L). Following separation of the aqueous and organic phases, the organic phase is distilled at 70° C. and atmospheric pressure to give (3S,5R)-3-cyano-5-methyl-octanoic acid ethyl ester as an oil (48.9 kg, 88% yield). ¹H NMR (400 MHz, CDCl₃) 4.17 ppm (q, J=7.83 Hz, 2H), 3.13-3.06 (m, 1H), 2.71-2.58 (m, 2H), 1.75-1.64 (m, 10H), 0.95 (d, J=6.34 3H), 0.92 (t, J=6.83, 3H, ¹³C NMR (CDCl3) 170.4, 121.8, 61.1, 39.6, 38.6, 37.0, 31.0, 25.9, 20.0, 18.5, 13.9.

### Example 12

### Preparation of (3S,5R)-3-aminomethyl-5-methyl-octanoic acid from (3S,5R)-3-cyano-5-methyl-octanoic acid ethyl ester

A solution (700 kg) containing (3S,5R)-3-cyano-5-methyl-octanoic acid ethyl ester (30%) in MTBE is treated with aqueous sodium hypochlorite solution (35 kg, 12%) and water (35 L). After stirring for 2 hours at RT, the mixture is allowed to settle for 3 hours, and the aqueous and organic phases are separated. The organic phase is washed with water (150 L) at RT and the mixture is allowed to separate into aqueous and organic phases. The organic phase is separated and subsequently reacted with NaOH aq (134 kg, 50%) and water (560 L). The reaction mixture is stirred for 2.5 h to 3.5 h at RT and the mixture is allowed to settle for 2 h. The resulting aqueous phase, which contains (3S,5R)-3-cyano-5-methyl-octanoic acid sodium salt, is fed to an autoclave which has been charged with sponge nickel A-7063 (43 kg) and purged with nitrogen. The autoclave is heated to 28° C. to 32° C. and is pressurized with hydrogen to 50 psig. The pressure is maintained at 50 psig for 18 h to 24 h. The autoclave is subsequently cooled to 20° C. to 30° C. and the pressure is reduced to 20 to 30 psig for sampling. The reaction is complete when the fractional conversion of (3S,5R)-3-cyano-5-methyl-octanoic acid sodium salt is 99% or greater. The reaction mixture is filtered and the filtrate is combined with an aqueous citric acid solution (64 kg in 136 kg of water) at a temperature of 20° C. to 30° C. Ethanol (310 L) is added and the mixture is heated to 55° C. to 60° C. The mixture is held for 1 h and then cooled at a rate of about −15° C./h until the mixture reaches at temperature of about 2° C. to 8° C. The mixture is stirred at that temperature for about 1.5 h and filtered. The resulting filter cake is rinsed with water (150 L) at 2° C. to 8° C. and then dried at RT with a nitrogen sweep until the water content is less than 1% by KF analysis, thus giving crude 3S,5R)-3-aminomethyl-5-methyl-octanoic acid.

The crude product (129 kg) is charged to a vessel. Water (774 kg) and anhydrous EtOH (774 kg) are added to the vessel and the resulting mixture is heated at reflux (about 80° C.) until the solution clears. The solution is passed through a polish filter (1μ) and is again heated at reflux until the solution clears. The solution is allowed to cool at a rate of about −20° C./h until it reaches a temperature of about 5° C., during which a precipitate forms. The resulting slurry is held at 0° C. to 5° C. for about 90 min to complete the crystallization process. The slurry is filtered to isolate the titled compound, which is rinsed with anhydrous EtOH (305 kg) and dried at under a nitrogen sweep at a temperature of 40° C. to about 45° C. until the water content (by KF) and the EtOH content (by GC) are each less than 0.5% by weight. Representative yield of the titled compound from (3S,5R)-3-cyano-5-methyl-octanoic acid ethyl ester is about 76%.

It should be noted that, as used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. It is to be understood that the above description is intended to be illustrative and not restrictive. Many embodiments will be apparent to those of skill in the art upon reading the above description. Therefore, the scope of the invention should be determined with references to the appended claims and includes the full scope of equivalents to which such claims are entitled. The disclosures of all articles and references, including patents, patent applications and publications, are herein incorporated by reference in their entirety and for all purposes.

What is claimed is:

1. A method of making a compound of Formula 1, 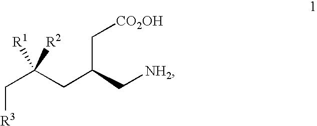
a diastereomer thereof, or pharmaceutically acceptable complex, salt, solvate or hydrate thereof, wherein
R¹ and R² are each independently selected from hydrogen atom and C₁₋₃ alkyl, provided that when R¹ is a hydrogen atom, R² is not a hydrogen atom;
R³ is selected from C₁₋₆ alkyl, C₂₋₆ alkenyl, C₃₋₆ cycloalkyl, C₃₋₆ cycloalkyl-C₁₋₆ alkyl, C₁₋₆ alkoxy, aryl, and aryl-C₁₋₃ alkyl, wherein each aryl moiety is optionally substituted with from one to three substituents independently selected from C₁₋₃ alkyl, C₁₋₃ alkoxy, amino, C₁₋₃ alkylamino, and halogeno; and
wherein each of the aforementioned alkyl, alkenyl, cycloalkyl, and alkoxy moieties are optionally substituted with from one to three fluorine atoms,
the method comprising:
(a) reducing a cyano moiety of a compound of Formula 8, 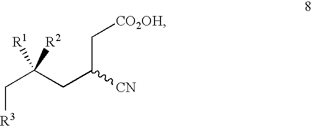
or a salt thereof to give a compound of Formula 9, 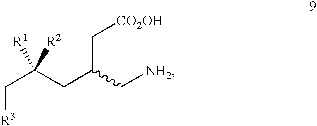
or a salt thereof, wherein R¹, R², and R³ in Formula 8 and Formula 9 are as defined for Formula 1;
(b) optionally treating a salt of the compound of Formula 9 with an acid;
(c) resolving the compound of Formula 9 or a salt thereof; and
(d) optionally converting the compound of Formula 1 or a salt thereof into a pharmaceutically acceptable complex, salt, solvate or hydrate thereof.

2. The method of claim 1, wherein reducing the cyano moiety comprises reacting the compound of Formula 8 or a salt thereof with hydrogen in the presence of a catalyst.

3. The method of claim 2, further comprising hydrolyzing a compound of Formula 7, 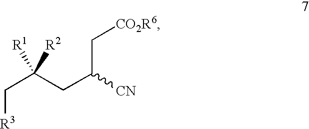
to give the compound of Formula 8 or a salt thereof, wherein
R¹, R², and R³ in Formula 7 are as defined for Formula 1, above; and
R⁶ is selected from C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl, wherein each of the aforementioned aryl moieties may be optionally substituted with from one to three substituents independently selected from C₁₋₃ alkyl, C₁₋₃ alkoxy, amino, C₁₋₃ alkylamino, and halogeno.

4. A method of making a compound of Formula 1, 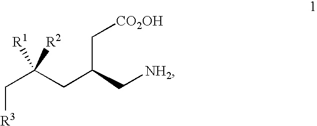
a diastereomer thereof, or pharmaceutically acceptable complex, salt, solvate or hydrate thereof, wherein
R¹ and R² are each independently selected from hydrogen atom and C₁₋₃ alkyl, provided that when R¹ is a hydrogen atom, R²is not a hydrogen atom;
R³ is selected from C₁₋₆ alkyl, C₂₋₆ alkenyl, C₃₋₆ cycloalkyl, C₃₋₆ cycloalkyl-C₁₋₆ alkyl, C₁₋₆ alkoxy, aryl, and aryl-C₁₋₃ alkyl, wherein each aryl moiety is optionally substituted with from one to three substituents independently selected from C₁₋₃ alkyl, C₁₋₃ alkoxy, amino, C₁₋₃ alkylamino, and halogeno; and
wherein each of the aforementioned alkyl, alkenyl, cycloalkyl, and alkoxy moieties are optionally substituted with from one to three fluorine atoms,
the method comprising:
(a) reducing a cyano moiety of a compound of Formula 12, 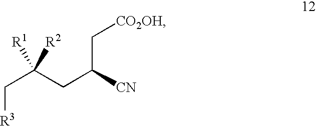
a diastereomer thereof, or a salt thereof, wherein R¹, R², and R³ in Formula 12 are as defined for Formula 1; and
(b) optionally converting the compound of Formula 1 or a salt thereof into a pharmaceutically acceptable complex, salt, solvate or hydrate thereof.

5. The method of claim 4, wherein reducing the cyano moiety comprises reacting the compound of Formula 12 or a salt thereof with hydrogen in the presence of a catalyst.

6. The method of claim 4, further comprising:
(a) contacting a compound of Formula 7, 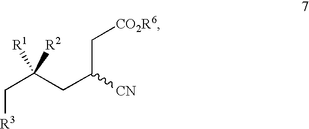
with an enzyme to yield the compound of Formula 10, 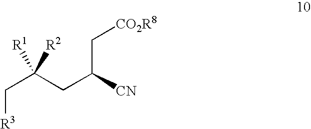
or a salt thereof, and a compound of Formula 11, 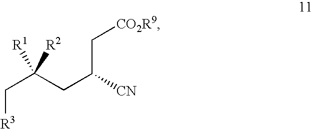
or a salt thereof, wherein the enzyme is adapted to diastereoselectively hydrolyze the compound of Formula 7 to the compound of Formula 10 or a salt thereof, or to a compound of Formula 11 or a salt thereof;
(b) isolating the compound of Formula 10, a diastereomer thereof, or a salt thereof; and
(c) optionally hydrolyzing the compound of Formula 10 or a diastereomer thereof, to give the compound of Formula 12, or a diastereomer thereof, wherein
R¹, R², and R³ in Formula 7, Formula 10, and Formula 11 are as defined for Formula 1, above;
R⁶ in Formula 7 is selected from C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl; and
R⁸ and R⁹ in Formula 10 and 11 are each independently selected from hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl;
wherein each of the aforementioned aryl moieties may be optionally substituted with from one to three substituents independently selected from C₁₋₃ alkyl, C₁₋₃ alkoxy, amino, C₁₋₃ alkylamino, and halogeno.

7. The method of claim 6, wherein R⁸ and R⁹ are independently selected from hydrogen atom and C₁₋₆ alkyl, provided that R⁸ and R⁹ are not both hydrogen atoms.

8. The method of claim 6, wherein R⁸ and R⁹ are independently selected from hydrogen atom, methyl, ethyl, n-propyl, and i-propyl, provided that R⁸ and R⁹ are not both hydrogen atoms.

9. The method of claim 8, wherein R⁹ is a hydrogen atom.

10. The method as in any one of claims 3, 6, 7, 8, and 9, wherein R⁶ is C₁₋₆ alkyl.

11. The method as in any one of claims 3, 6, 7, 8, and 9, wherein R⁶ is methyl, ethyl, n-propyl or i-propyl.

12. The method as in any one of claims 1 to 11, wherein R¹ and R² are each independently hydrogen or methyl, provided that R¹ and R²are not both hydrogen atoms, and R³ is C₁₋₆ alkyl.

13. The method as in any one of claims 1 to 11, wherein R¹ is hydrogen, R² is methyl, and R³ is methyl, ethyl, n-propyl or i-propyl.

14. The method as in any one of claims 1 to 11, wherein R¹ is hydrogen, R² is methyl, and R³ is ethyl.

15. A compound of Formula 19, 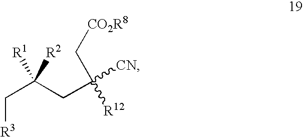
including salts thereof, wherein
R¹ and R² are each independently selected from hydrogen atom and C₁₋₃ alkyl, provided that when R¹ is a hydrogen atom, R² is not a hydrogen atom;
R³ is selected from C₁₋₆ alkyl, C₂₋₆ alkenyl, C₃₋₆ cycloalkyl, C₃₋₆ cycloalkyl-C₁₋₆ alkyl, C₁₋₆ alkoxy, aryl, and aryl-C₁₋₃ alkyl;
R⁸ is selected from hydrogen atom, C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl;
R¹² is a hydrogen atom or —C(O)OR⁷; and
R⁷ is selected from C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl;
wherein each of the aforementioned aryl moieties is optionally substituted with from one to three substituents independently selected from C₁₋₃ alkyl, C₁₋3 alkoxy, amino, C₁₋₃ alkylamino, and halogeno; and
wherein each of the aforementioned alkyl, alkenyl, cycloalkyl, and alkoxy moieties are optionally substituted with from one to three fluorine atoms.

16. The compound of claim 15, wherein R⁷ is C₁₋₆ alkyl.

17. The compound of claim 15, wherein R⁷ is methyl, ethyl, n-propyl or i-propyl.

18. The compound of claim 15 which is given by Formula 7, 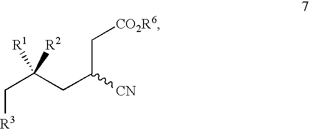
wherein
R¹, R², and R³ are as defined for Formula 19, above; and
R⁶ is selected from C₁₋₆ alkyl, C₂₋₆ alkenyl, C₂₋₆ alkynyl, C₃₋₇ cycloalkyl, C₃₋₇ cycloalkenyl, halo-C₁₋₆ alkyl, halo-C₂₋₆ alkenyl, halo-C₂₋₆ alkynyl, aryl-C₁₋₆ alkyl, aryl-C₂₋₆ alkenyl, and aryl-C₂₋₆ alkynyl;
wherein each of the aforementioned aryl moieties is optionally substituted with from one to three substituents independently selected from C₁₋₃ alkyl, C₁₋₃ alkoxy, amino, C₁₋₃ alkylamino, and halogeno; and
wherein each of the aforementioned alkyl, alkenyl, cycloalkyl, and alkoxy moieties are optionally substituted with from one to three fluorine atoms.

19. The compound of claim 18, wherein R⁶ is C₁₋₆ alkyl.

20. The compound of claim 18, wherein R⁶ is methyl, ethyl, n-propyl or i-propyl.

21. The compound of claim 15 which is given by Formula 8, 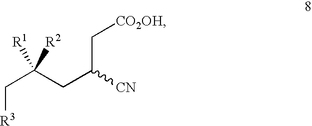
or a salt thereof, wherein R¹, R², and R³ are as defined for Formula 19, above.

22. The compound of claim 15 which is given by Formula 10, 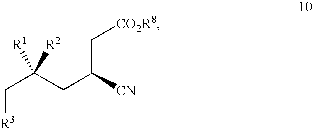
a diastereomer thereof, or a salt thereof, wherein R¹, R², R³, and R⁸ are as defined for Formula 19, above.

23. The compound of claim 22, wherein R⁸ is selected from hydrogen atom and C₁₋₆ alkyl.

24. The compound of claim 22, wherein R⁸ is selected from hydrogen atom, methyl, ethyl, n-propyl, and i-propyl.

25. The compound of claim 15 which is given by Formula 12, 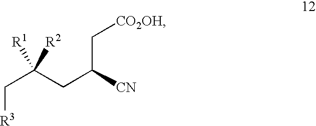
a diastereomer thereof, or a salt thereof, wherein R¹, R², and R³ are as defined for Formula 19, above.

26. The compound as in any one of claims 15 to 25, wherein R¹ and R² are each independently hydrogen or methyl, provided that R¹ and R² are not both hydrogen atoms, and R³ is C₁₋₆ alkyl.

27. The compound as in any one of claims 15 to 25, wherein R¹ is hydrogen, R² is methyl, and R³ is methyl, ethyl, n-propyl or i-propyl.

28. The compound as in any one of claims 15 to 25, wherein R¹ is hydrogen, R² is methyl, and R³ is ethyl.

29. The compound of claim 15, selected from:
(2′R)-2-cyano-2-(2′-methyl-butyl)-succinic acid diethyl ester;
(2′R)-2-cyano-2-(2′-methyl-pentyl)-succinic acid diethyl ester;
(2′R)-2-cyano-2-(2′-methyl-hexyl)-succinic acid diethyl ester;
(2′R)-2-cyano-2-(2′,4′-dimethyl-pentyl)-succinic acid diethyl ester;
(5R)-3-cyano-5-m ethyl-heptanoic acid ethyl ester;
(5R)-3-cyano-5-methyl-octanoic acid ethyl ester;
(5R)-3-cyano-5-methyl-nonanoic acid ethyl ester;
(5R)-3-cyano-5,7-dimethyl-octanoic acid ethyl ester;
(5R)-3-cyano-5-methyl-heptanoic acid;
(5R)-3-cyano-5-methyl-octanoic acid;
(5R)-3-cyano-5-methyl-nonanoic acid;
(5R)-3-cyano-5,7-dimethyl-octanoic acid;
(3S,5R)-3-cyano-5-methyl-heptanoic acid;
(3S,5R)-3-cyano-5-methyl-octanoic acid;
(3S,5R)-3-cyano-5-methyl-nonanoic acid;
(3S,5R)-3-cyano-5,7-dimethyl-octanoic acid;
(3S,5R)-3-cyano-5-methyl-heptanoic acid ethyl ester;
(3S,5R)-3-cyano-5-methyl-octanoic acid ethyl ester;
(3S,5R)-3-cyano-5-methyl-nonanoic acid ethyl ester;
(3S,5R)-3-cyano-5,7-dimethyl-octanoic acid ethyl ester;
(3R,5R)-3-cyano-5-methyl-heptanoic acid;
(3R,5R)-3-cyano-5-methyl-octanoic acid;
(3R,5R)-3-cyano-5-methyl-nonanoic acid;
(3R,5R)-3-cyano-5,7-dimethyl-octanoic acid;
(3R,5R)-3-cyano-5-methyl-heptanoic acid ethyl ester;
(3R,5R)-3-cyano-5-methyl-octanoic acid ethyl ester;
(3R,5R)-3-cyano-5-methyl-nonanoic acid ethyl ester;
(3R,5R)-3-cyano-5,7-dimethyl-octanoic acid ethyl ester; and
diastereomers and opposite enantiomers of the aforementioned compounds, and salts of the aforementioned compounds, their diastereomers and opposite enantiomers.
